(19) United States
(12) Patent Application Publication
(10) Pub. No.: US 2020/0340897 A1
(43) Pub. Date: Oct. 29, 2020

(54) DEVICES AND METHODS FOR MONITORING LIQUID-SOLID CONTACT TIME

(71) Applicant: Essenlix Corporation, Monmouth Junction, NJ (US)

(72) Inventors: Stephen CHOU, Princeton, NJ (US); Wei DING, East Windsor, NJ (US)

(73) Assignee: Essenlix Corporation, Monmouth Junction, NJ (US)

(21) Appl. No.: 16/759,106

(22) PCT Filed: Oct. 26, 2018
(86) PCT No.: PCT/US18/57849

§ 371 Date: Apr. 24, 2020

Related U.S. Application Data
(60) Provisional application No. 62/577,339, filed on Oct. 26, 2017.

(51) Int. Cl. G01N 13/00; G01N 33/487; G01N 1/38; G01N 1/28

(52) U.S. Cl. CPC G01N 13/00; G01N 33/487; G01N 2001/388; G01N 1/2813; G01N 2013/003; G01N 1/38

(57) ABSTRACT

Among other things, the present invention is related to the field of bio/chemical sampling, sensing, assays and other applications.

### CROSS-REFERENCE

This application is a National Stage entry (§ 371) application of International Application No. PCT/US18/57849, filed on Oct. 26, 2018, which claims the benefit of U.S. Provisional Patent Application No. 62/577,339, filed Oct. 26, 2017, the contents of which are relied upon and incorporated herein by reference in their entirety.

The entire disclosure of any publication or patent document mentioned herein is entirely incorporated by reference.

### FIELD OF THE INVENTION

Among other things, the present invention is related to the field of bio/chemical sampling, sensing, assays and other applications.

### BACKGROUND

In testing an analyte in a sample using a plate that has a regent placed on the surface of the plate, there is a need to know the time between the time that the sample begins to contact the surface and the time that a measurement is being made. The present invention offers the devices, systems and methods for such need.

### BRIEF SUMMARY

In certain embodiments of the present disclosure, a device for analyzing a thin layer sample can comprise a first plate and a diffusion marker. In certain embodiments, the first plate has a sample contact area on its inner surface for contacting a thin layer sample of a thickness of 1 mm or less. In certain embodiments, the diffusion marker is positioned in the sample contact area of the first plate and is configured to, upon contacting the sample, diffuse in the sample with a pre-determined diffusion rate. In certain embodiments, the diffusion marker is distinguishable from the sample when diffusing in the sample. In certain embodiments, the diffusion of the diffusion marker indicates a time duration that the sample is in contact with the first plate inner surface.

In certain embodiments of the present disclosure, a device for analyzing a liquid sample can comprise a first plate, a second plate, spacers, and a diffusion marker. In certain embodiments, the plates are movable relative to each other into different configurations. In certain embodiments, one or both plates are flexible. In certain embodiments, both plates have, on its respective inner surface, a sample contact area for contacting a sample. In certain embodiments, the spacers are fixed to the respective inner surface of one or both of the plates and have a predetermined substantially uniform height. In certain embodiments, the diffusion marker is positioned in the sample contact area of one or both of the plates and is configured to, upon contacting the sample, diffuse in the sample with a pre-determined diffusion rate. In certain embodiments, the diffusion marker is distinguishable from the sample when diffusing in the sample. In certain embodiments, one of the configurations is an open configuration, in which: the two plates are partially or entirely separated apart, the spacing between the plates is not regulated by the spacers, and the sample is deposited on one or both of the plates. In certain embodiments, another of the configurations is a closed configuration, which is configured after the sample deposition in the open configuration, and in the closed configuration: at least part of the deposited sample is compressed by the two plates into a layer of uniform thickness that is confined by the two plates, and the uniform thickness of the layer is regulated by the plates and the spacers, wherein the sample thickness is 1 mm or less. In certain embodiments, the diffusion of the diffusion marker indicates a time duration that the sample is in contact with the respective inner surface of the plate on which the diffusion marker is positioned.

In certain embodiments, the diffusion marker is one single entity and diffuses in one direction in the sample, and wherein a diffusion distance of the diffusion marker indicates the time duration that the sample is in contact with the respective plate on which the diffusion marker is positioned. In certain embodiments, the diffusion marker comprises a plurality of entities that all diffuse in one direction in the sample, and wherein a diffusion distance of the diffusion marker indicates the time duration that the sample is in contact with the respective plate on which the diffusion marker is positioned. In certain embodiments, the diffusion marker comprises a plurality of entities, and the diffusion marker as a whole diffuses in more than one direction in the sample, and wherein at least one dimension of the diffusion marker indicates the time duration that the sample is in contact with the respective plate on which the diffusion marker is positioned. In certain embodiments, the diffusion marker diffuses isotropically in the more than one diffusion direction in the liquid solution. In certain embodiments, the diffusion marker diffuses anisotropically in the more than one diffusion direction in the liquid solution. In certain embodiments, the diffusion marker is confined by a physical barrier thereby preventing the diffusion marker from diffusing in one or more directions in the liquid solution. In certain embodiments, the diffusion marker is confined to diffuse in a groove on the inner surface of the plate on which the diffusion marker is positioned. In certain embodiments, the diffusion marker, when diffusing in the sample, is distinguishable from the sample by at least one parameter of the diffusion marker that is selected from the group consisting of light absorption, reflection, transmission, diffraction, scattering, and diffusion; luminescence, heat, viscosity, and magnetism. In certain embodiments, the predetermined diffusion rate is constant. In certain embodiments, the predetermined diffusion rate is a predetermined function of time.

In certain embodiments of the present disclosure, a method of analyzing a liquid sample can comprise obtaining a device of any prior claim. In certain embodiments of the present disclosure, a method of analyzing a liquid sample can comprise depositing the liquid sample on one or both of the plates of the device at the open configuration. In certain embodiments of the present disclosure, a method of analyzing a liquid sample can comprise bringing the two plates together and compressing the plates into the closed configuration. In certain embodiments of the present disclosure, a method of analyzing a liquid sample can comprise, at the closed configuration, analyzing the liquid sample at a time point. In certain embodiments of the present disclosure, a method of analyzing a liquid sample can comprise determining at the time point the time duration that the sample is deposited on one or both of the plates by monitoring the diffusion of the diffusion marker in the deposited sample.

In certain embodiments of the present disclosure, a device for analyzing a liquid sample can comprise a first plate and an interaction marker. In certain embodiments, the first plate has a sample contact area on its inner surface for contacting the sample. In certain embodiments, the interaction marker is positioned in the sample contact area of the first plate and is configured to, upon contacting the sample, interact with the sample to bring about an interaction signal. In certain embodiments, the interaction signal is configured to indicate the time duration that the sample is in contact with the first plate inner surface.

In certain embodiments of the present disclosure, a device for analyzing a liquid sample can comprise a first plate, a second plate, spacers, and an interaction marker. In certain embodiments, the plates are movable relative to each other into different configurations. In certain embodiments, one or both plates are flexible. In certain embodiments, both plates have, on its respective inner surface, a sample contact area for contacting a sample. In certain embodiments, the spacers are fixed to the respective inner surface of one or both of the plates and have a predetermined substantially uniform height. In certain embodiments, the interaction marker is positioned in the sample contact area of one or both of the plates and is configured to, upon contacting the sample, interact with the sample to bring about an interaction signal. In certain embodiments, one of the configurations is an open configuration, in which: the two plates are partially or entirely separated apart, the spacing between the plates is not regulated by the spacers, and the sample is deposited on one or both of the plates. In certain embodiments, another of the configurations is a closed configuration, which is configured after the sample deposition in the open configuration, and in the closed configuration: at least part of the deposited sample is compressed by the two plates into a layer of uniform thickness that is confined by the two plates, and the uniform thickness of the layer is regulated by the plates and the spacers. In certain embodiments, the interaction signal is configured to indicate the time duration that the sample is in contact with the respective inner surface of the plate on which the interaction marker is positioned. In certain embodiments, the interaction marker comprises of a plurality of entities of one species. In certain embodiments, the interaction marker comprises of a plurality of entities of different species, and the entities of different species are located on the inner surfaces of different plates. In certain embodiments, the interaction signal is provided by the interaction marker. In certain embodiments, the interaction signal is provided by the sample. In certain embodiments, the interaction signal is provided by an external entity. In certain embodiments, the interaction signal is in a signal form selected from the group consisting of: chromatic signal, luminescence signal, heat, magnetic signal, electric signal, sound, any other electromagnetic signals, and any combination thereof.

In certain embodiments of the present disclosure, a method of analyzing a liquid sample can comprise obtaining a device of any prior claim. In certain embodiments of the present disclosure, a method of analyzing a liquid sample can comprise depositing the liquid sample on one or both of the plates of the device at the open configuration. In certain embodiments of the present disclosure, a method of analyzing a liquid sample can comprise bringing the two plates together and compressing the plates into the closed configuration. In certain embodiments of the present disclosure, a method of analyzing a liquid sample can comprise, at the closed configuration, analyzing the liquid sample at a time point. In certain embodiments of the present disclosure, a method of analyzing a liquid sample can comprise determining at the time point the time duration that the sample is deposited on one or both of the plates by monitoring the interaction signal.

In certain embodiments, the determining is performed using at least one optical imaging method. In certain embodiments, the layer of uniform thickness limits the diffusion of the interaction marker to 1 dimension or 2 dimensions. In certain embodiments, the interaction marker is a dye, and wherein the dye changes color upon contacting the sample. In certain embodiments, the diffusion marker is an optical label. In certain embodiments, the method is performed under imperfect conditions. In certain embodiments, artificial intelligence and/or machine learning is used to accurately determine the time duration. In certain embodiments, the images taken during an assay operation and/or the samples measured by an assay are analyzed by artificial intelligence and machine learning. In certain embodiments, the samples are selected from the group consisting of medical samples, biology samples, environmental samples and chemistry samples. In certain embodiments, the sample is held by a QMAX device. In certain embodiments, the embodiment can further comprise providing a machine learning framework to enhance the functionality, application scope and/or the accuracy in assaying using QMAX device.

In certain embodiments of the present disclosure, a method for assaying sample and/or assay operation (e.g. diffusion maker's diffusion time) that utilizes QMAX together with imaging plus a machine learning and/or artificial intelligence can comprise using a QMAX device that has an auxiliary structure in the form of pillars to precisely control the distribution and volume of the sample in assaying, wherein the sample for assaying is loaded into the QMAX device and is kept between the two parallel plates on the QMAX device with an upper plate being transparent for imaging by an imager. In certain embodiments of the present disclosure, a method for assaying sample and/or assay operation (e.g. diffusion maker's diffusion time) that utilizes QMAX together with imaging plus a machine learning and/or artificial intelligence can comprise the gap between the two parallel plates in the QMAX device is spaced narrowly—with the distance of the gap being proportional to the size of the analytes to be assayed—by which the analytes in the sample form a single layer between the said plates that can be imaged by an imager on the QMAX device. In certain embodiments of the present disclosure, a method for assaying sample and/or assay operation (e.g. diffusion maker's diffusion time) that utilizes QMAX together with imaging plus a machine learning and/or artificial intelligence can comprise the sample volume corresponding to the Aol (area-of-interest) on the upper plate of the QMAX device can be precisely characterized by Aol and the gap—because of the uniformity of the gap between the plates in the QMAX device. In certain embodiments of the present disclosure, a method for assaying sample and/or assay operation (e.g. diffusion maker's diffusion time) that utilizes QMAX together with imaging plus a machine learning and/or artificial intelligence can comprise the image on the sample for assaying sandwiched between the Aol x gap in the QMAX device is a pseudo-2D image, because it has the appearance of a 2D image, but it is an image of a 3D sample with its depth being known priori or characterized through other means. In certain embodiments of the present disclosure, a method for assaying sample and/or assay operation (e.g. diffusion maker's diffusion time) that utilizes QMAX together with imaging plus a machine learning and/or artificial intelligence can comprise the captured pseudo-2D sample image taken over the Aol of the QMAX device can characterize the location of the analytes, color, shape, counts, and concentration of the analytes in the sample for assaying. In certain embodiments of the present disclosure, a method for assaying sample and/or assay operation (e.g. diffusion maker's diffusion time) that utilizes QMAX together with imaging plus a machine learning and/or artificial intelligence can comprise based on abovementioned properties, the captured pseudo-2D image of QMAX device for assaying is amendable to a machine learning framework that applies to analyte detection, localization, identification, segmentation, counting, etc. for assaying in various applications. In certain embodiments of the present disclosure, a method for assaying sample and/or assay operation (e.g. diffusion maker's diffusion time) that utilizes QMAX together with imaging plus a machine learning and/or artificial intelligence can comprise any combination of thereof.

In certain embodiments, the embodiment further comprises implementing a machine learning framework for QMAX based devices into a device that is capable of running an algorithm such as deep learning to discriminatively locate, identify, segment and count analytes based on the pseudo-2D image captured by the QMAX imager. In certain embodiments, the machine learning improves the images captured by the imager on the QMAX device and reduces the effects of noise and artifacts—including and not limited to air bobbles, dusts, shadows, and pillars. In certain embodiments, the training of machine learning uses the spacers of the QMAX card to reduce the data size of training set.

### BRIEF DESCRIPTION OF FIGURES

The skilled artisan will understand that the drawings, described below, are for illustration purposes only. The drawings are not intended to limit the scope of the present teachings in any way. The drawings may not be in scale. In the figures that present experimental data points, the lines that connect the data points are for guiding a viewing of the data only and have no other meaning.

FIG. 1 schematically illustrates an exemplary embodiment of the device and method according to the present invention, which a diffusion marker is used to monitor the contact duration.

FIG. 2 schematically illustrates another exemplary embodiment of the device and method according to the present invention, in which a diffusion barrier is used to physically confine the diffusion of the diffusion marker to the lateral plane.

FIG. 3 schematically illustrates another exemplary embodiment of the device and method according to the present invention, in which a diffusion marker is used to monitor the contact duration.

FIG. 4 shows experimental results using an exemplary embodiment of the device and method provided by the present invention for monitoring, in which a mixture of glucose assay reagents was used as an exemplary interaction marker.

FIG. 5 is an illustration of a CROF (Compressed Regulated Open Flow) embodiment. Panel (a) illustrates a first plate and a second plate wherein the first plate has spacers. Panel (b) illustrates depositing a sample on the first plate (shown), or the second plate (not shown), or both (not shown) at an open configuration. Panel (c) illustrates (i) using the two plates to spread the sample (the sample flow between the plates) and reduce the sample thickness, and (ii) using the spacers and the plate to regulate the sample thickness at the closed configuration. The inner surface of each plate may have one or a plurality of binding sites and or storage sites (not shown).

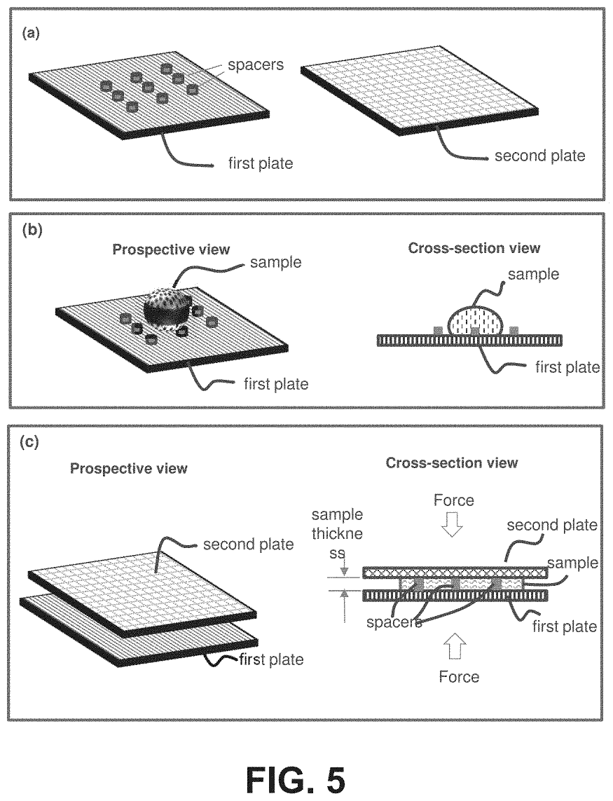

### DESCRIPTION OF EXEMPLARY EMBODIMENTS

The following detailed description illustrates some embodiments of the invention by way of example and not by way of limitation. The section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

The present invention is related to, among other things, methods, devices, and systems that can improve and/or speed up the quantification, binding, and/or sensing of an analyte and/or entity in a sample.

The term “diffusion marker” refers to a substance (a) that is capable of diffuse in a sample under a testing and (b) its diffusion is used to monitor the time duration between the time that a sample begins in contact with the marker and a time after the beginning of the contact.

The term “interaction marker” refers to a substance, wherein the substance is capable of interacting with a sample, upon in contacting with the sample, to generate a product substance and the product substance is capable of being used as a diffusion marker.

### 1. DIFFUSION MARKER AS A CONTACT INDICATOR

One aspect of the present invention provides the device for monitoring of the time duration that a liquid and a solid are in contact, which, in some embodiments, monitors a diffusion of a diffusion maker in the liquid, wherein the diffusion marker is placed on a location of the solid surface before the liquid contacts the solid surface and configured to, upon contacting the liquid, diffuse with a predetermined diffusion rate in the liquid.

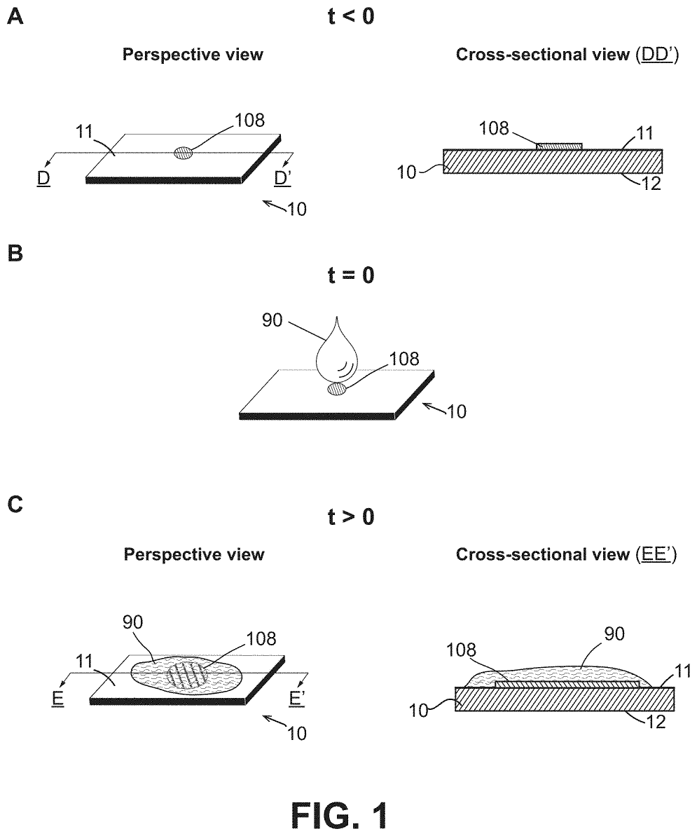

FIG. 1 schematically illustrates an exemplary embodiment of the device and method according to the present invention. Specifically, as shown in the figure, the exemplary device comprises a first plate 10 and a diffusion marker 108. The first plate 10 comprises an inner surface 11 and an outer surface 12. The diffusion marker 108 is positioned on the inner surface 11 of the first plate 10. The diffusion marker 108 is distinguishable from the liquid solution and configured to, upon contacting a liquid solution 90, diffuse in the liquid solution. The term “distinguishable” as used herein describes an object that can be distinguished from other objects that said object is compared with, based on its one or more distinct characteristics that can be detected by certain approaches. In certain embodiments, the distinguishing is by an optical imaging methods. For instance, in this particular exemplary case, the diffusion marker 108 has a color different from the liquid solution that remains distinct while it diffuses in the liquid solution, thus it is visually distinguishable from the liquid solution 90. Panel (A) shows both perspective and cross-sectional views of the exemplary device before the diffusion marker 108 contacting the liquid solution 90 (time t<0). Panel (B) shows the time point when the diffusion marker 108 contacts the liquid solution 90 (time t=0). Panel (C) shows both perspective and cross-sectional views of the exemplary device after the diffusion marker 108 having contacted the liquid solution 90 for a period of time (time t>0). In this exemplary case, the diffusion marker 108 diffuses laterally in the liquid solution. The term “lateral” as used herein refers to any orientation, motion direction, or structural configuration that is on a plane parallel to the inner surface of the first plate of the device. As shown in the figure, the lateral dimension of the diffusion marker 108 is increased at t>0 as compared to t<0, as a result of the lateral diffusion of the diffusion marker after its contact of the liquid solution 90. The term “lateral diffusion” as used herein refers to the diffusion at any direction that is parallel to the inner surface of the first plate of the device.

### 1.1. Diffusion Direction and Diffusion Rate

In some embodiments, the diffusion marker is one single entity. In some embodiments, the diffusion marker is a mixture, combination, or group of a plurality of entities.

In some embodiments, the diffusion marker is one single entity and diffuses in only one direction in the liquid solution.

In some embodiments, the diffusion marker is a mixture, combination, or group of a plurality of entities, and diffuses in only one direction in the liquid solution, meaning that all the constituent entities of the diffusion marker diffuse in one single direction.

In some embodiments, the diffusion marker is a mixture, combination, or group of a plurality of entities, and diffuses in more than one direction in the liquid solution, meaning that the constituent entities of the diffusion marker as a whole diffuse in more than one direction. In these embodiments, each individual constituent entity could have different diffusion directions, but the diffusion marker as a whole enlarges its dimension along said one or more direction in the liquid solution. The term “dimension” as used herein in the context of the diffusion marker measures the furthest distance between two constituent entities of the diffusion marker along said more than one direction.

In some embodiments, as discussed above in FIG. 1, the diffusion marker is configured to, upon contacting the liquid solution, diffuse laterally in it. In some embodiments, the diffusion marker is configured to, upon contacting the liquid solution, diffuse along any other plane than the lateral plane in the liquid solution. In some embodiments, the diffusion marker is configured to, upon contacting the liquid solution, diffuse along more than one plane in the liquid solution.

In some embodiments, the diffusion marker is configured to, upon contacting the liquid solution, diffuse in only one single direction. In some embodiments, the diffusion marker is configured to, upon contacting the liquid solution, diffuse in more than one directions in the liquid solution. In some embodiments, the diffusion marker is configured to, upon contacting the liquid solution, diffuse in two, three, four, five, six, or any number of directions. In some embodiments, the diffusion marker is configured to, upon contacting the liquid solution, diffuse in all directions in the liquid solution, if not encountering any barrier.

The term “diffuse with a predetermined rate” as used herein refers to the fact that the diffusion rate of the diffusion marker in one or more diffusion directions in the liquid solution is predetermined. In some embodiments, the diffusion marker comprises more than one entity. The constituent entities of the diffusion marker do not necessarily have a same predetermined rate in any diffusion direction, but it is a characteristic of the diffusion marker as a whole to have a predetermined rate.

In some embodiments, the diffusion marker only diffuses in one single direction in the liquid solution, and such diffusion is configured to have a predetermined rate. This means that the diffusion distance of the diffusion marker along this diffusion direction is proportional to the time that the diffusion marker is in contact with the liquid solution, at least within a certain time window. In these embodiments, the diffusion distance of the diffusion marker in the liquid solution is taken as an indicator of the contact duration between the solid substrate (i.e. the first plate 10) and the liquid solution (“contact duration” hereafter).

In some embodiments, more than one diffusion direction of the diffusion marker has a predetermine rate, such that the change in the dimension of the diffusion marker along any two of said more than one diffusion direction is proportional to the time duration that the diffusion marker is in contact with the liquid solution. In these embodiments, the dimensional change of the diffusion marker along said one or more diffusion direction is taken as an indicator of the contact duration. As exemplified in FIG. 1, the changes in any lateral dimension of the diffusion marker 108 can be taken as an indicator of the contact duration.

In some embodiments, the diffusion marker is configured to diffuse isotropically in the liquid solution. In some embodiments, the diffusion marker is configured to diffuse anisotropically in the liquid solution. In some embodiments, the anisotropic lateral diffusion of the diffusion marker is configured to have one or more diffusion direction that has a predetermined diffusion rate, such that monitoring said one or more diffusion direction gives a reading of the contact duration.

### 1.2. Position and Confinement of the Diffusion Marker

In some embodiments, the diffusion marker is configured to be detachable from the inner surface of the first plate upon contacting the liquid solution and freely diffuse in the liquid solution.

In some embodiments, the diffusion marker is chemically attached to the inner surface of the first plate such that it is only capable of diffusing on the inner surface of the first plate (i.e. molecules of the diffusion marker moving laterally on the inner surface while remaining attached thereto). Such chemical attachments include, but not limited to, covalent bonds, ionic bonds, metallic bonds, hydrogen bonds, and van der Waals bonds.

In some embodiments, the diffusion marker is physically limited to diffuse only laterally on the inner surface of the first plate. For instance, the diffusion marker is a hydrophobic agent that has extremely low solubility or intermiscibility (if the diffusion marker is a liquid agent) with the water-based liquid solution that is applied upon the first plate. Such a diffusion marker is configured to stay on top of the inner surface but at the bottom of the liquid solution and its diffusion only takes place laterally.

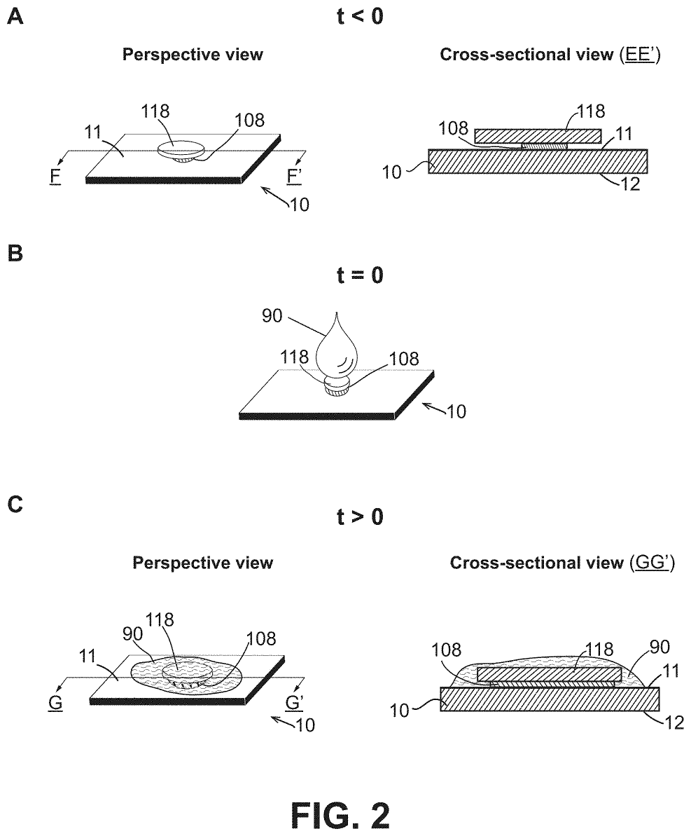

FIG. 2 schematically illustrates another exemplary embodiment of the device and method according to the present invention, which uses a diffusion barrier to physically confine the diffusion of the diffusion marker to the lateral plane. Similar to FIG. 1, the exemplary device comprises a first plate 10 and a diffusion marker 108. Additionally, it also comprises a diffusion barrier 118. The first plate 10 comprises an inner surface 11 and an outer surface 12. The diffusion marker 108 is positioned on the inner surface 11 of the first plate 10. The diffusion marker 108 is configured to, upon contacting a liquid solution, laterally diffuse in the liquid solution, while being distinguishable from the liquid solution. The diffusion barrier 118 is positioned on top of the diffusion marker 108, which is configured to block the diffusion of the diffusion marker 108 in the direction away from the first plate 10, such that it only diffuses laterally on the inner surface 11 of the first plate 10. Panel (A) shows both perspective and cross-sectional views of the exemplary device before the diffusion marker 108 contacting a liquid solution (time t<0). Panel (B) shows the time point when the diffusion marker 108 contacts a liquid solution 90 (time t=0). Panel (C) shows both perspective and cross-sectional views of the exemplary device after the diffusion marker 108 having contacted the liquid solution 90 for a period of time (time t>0). As shown in the figure, the lateral dimension of the diffusion marker 108 is increased at t>0 as compared to t<0, as a result of the lateral diffusion of the diffusion marker after its contact of the liquid solution 90.

In some embodiments, the diffusion marker is allowed to diffuse in any direction on the lateral plane. In some embodiments, the diffusion marker is confined to diffuse only in one or more directions on the lateral plane. For instance, in some embodiments, the diffusion marker is placed inside a linear groove on the inner surface of the firs plate, such that the diffusion marker can only diffuse inside and long the linear groove. In some embodiments, the groove is in an open shape with two or more ends, such as, but not limited to, straight line, a curved line, arc, tree-like shape with branches, and the like, and any other variations thereof. In some embodiments, the groove is in a closed shape with no ends, such as, but not limited to, circle, eclipse, triangle, rectangle, pentagon, hexagon, heptagon, octagon, enneagon, decagon, star, and the like, and any other variations thereof. For instance, in some design, the groove is circular, and the diffusion marker is configured to diffuse from one start point in either clockwise or counterclockwise direction along the circular groove, mimicking the circular movement of a clock finger.

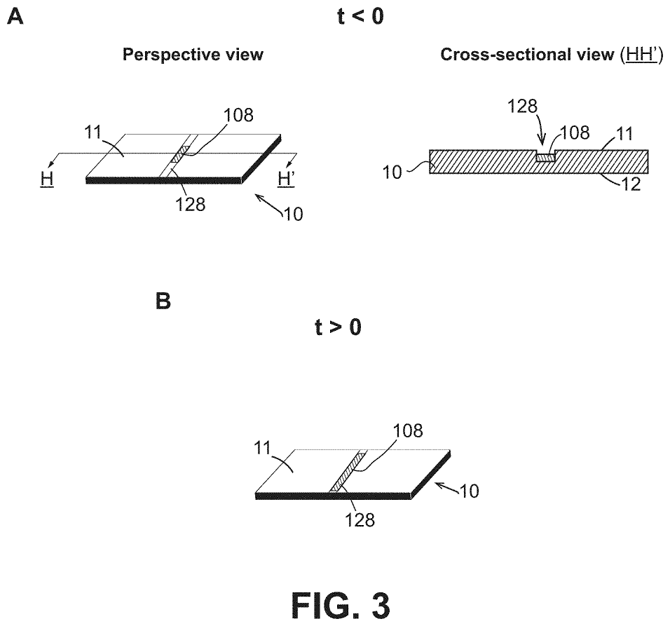

FIG. 3 schematically illustrates another exemplary embodiment of the device and method according to the present invention. Similar to FIG. 1, the exemplary device comprises a first plate 10 and a diffusion marker 108. Additionally, it also comprises a diffusion groove 128. The first plate 10 comprises an inner surface 11 and an outer surface 12. The diffusion marker 108 is positioned inside the diffusion groove 128 on the inner surface 11 of the first plate 10. The diffusion marker 108 is configured to, upon contacting a liquid solution, diffuse in the liquid solution, while being distinguishable from the liquid solution. The diffusion groove 128 is configured to contain the diffusion marker 108 and confine its diffusion inside the diffusion groove 128 with no or little diffusion away from the diffusion groove 128, such that it only diffuses laterally on the inner surface 11 of the first plate 10. Panel (A) shows both perspective and cross-sectional views (FF′) of the exemplary device before the diffusion marker 108 contacting a liquid solution (time t<0). Panel (C) shows a perspective view of the exemplary device after the diffusion marker 108 having contacted the liquid solution 90 for a period of time (time t>0). As shown in the figure, the lateral dimension along of the diffusion marker 108 the diffusion groove 128 is increased at t>0 as compared to t<0, as a result of the lateral diffusion of the diffusion marker after its contact of the liquid solution 90.

In some embodiments, on top of the diffusion maker 108 and the diffusion groove 128 there exists a diffusion barrier (not shown) capable of confining the diffusion of the diffusion marker to the lateral plane while at the same time allowing access of the liquid solution to the diffusion marker inside the groove. For instance, the barrier can have small channels for liquid solution to flow into the groove or can be limited to cover the groove only partially without losing the confinement of the diffusion marker. However, in some embodiments, there is no diffusion barrier on top of the diffusion groove 128, rather the diffusion groove 128 is designed to have very small opening such that the liquid solution that flows into the groove is restricted from flowing outward due to factors such as, but not limited to, capillary force and surface tension, and thus the diffusion marker 108 is blocked from diffusing out of the groove 128.

In some embodiments, there is only one diffusion marker on the inner surface of the first plate. In other embodiments, there are more than one diffusion maker positioned at more than one locations on the inner surface of the first plate.

### 2. USE OF DIFFUSION MARKER IN COF AND CROF PROCESS

Another aspect of the present invention provides a COF device and method that use the diffusion marker to monitor the contact duration as discussed above. Yet another aspect of the present invention provides a CROF device and method that use the diffusion marker to monitor the contact duration as discussed above.

The term “compressed open flow (COF)” refers to a method that changes the shape of a flowable sample deposited on a plate by (i) placing other plate on top of at least a part of the sample and (ii) then compressing the sample between the two plates by pushing the two plates towards each other; wherein the compression reduces a thickness of at least a part of the sample and makes the sample flow into open spaces between the plates. The term “compressed regulated open flow” or “CROF” (or “self-calibrated compressed open flow” or “SCOF” or “SCCOF”) (also known as QMAX) refers to a particular type of COF, wherein the final thickness of a part or entire sample after the compression is “regulated” by spacers, wherein the spacers are placed between the two plates. Here the CROF device is used interchangeably with the QMAX device.

A COF device and a CROF device as described here include but not limited to the COF and QMAX device, respectively, described in U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054025, which was filed on Sep. 27, 2016, the complete disclosures of which are hereby incorporated by reference for all purposes.

In some embodiments, a COF device comprises a first plate and a second plate. The two plates are movable relatively to each other into different configurations, including an open configuration and a closed configuration. In the open configuration, the two plates are partially or entirely separated apart, and a sample is deposited on one or both of the plates. The closed configuration is configured after the sample deposition in the open configuration, and in the closed configuration, at least part of the sample is compressed by the two plates into a thin layer as compared to the sample in the open configuration.

In some embodiments, a CROF device comprises a first plate, a second plate, and spacers. The spacers are fixed to one or both of the plates. The two plates are movable relatively to each other into different configurations, including an open configuration and a closed configuration. In the open configuration, the two plates are partially or entirely separated apart, the spacing between the two plates is not regulated by the spacers, and a sample is deposited on one or both of the plates. The closed configuration is configured after the sample deposition in the open configuration, and in the closed configuration, at least part of the sample is compressed by the two plates into a thin layer, the thickness of the thin layer is confined by the two plates and is regulated by the spacers and the plates.

In some embodiments, the COF or CROF device further comprises a diffusion marker that is placed on top of either one or both of the plates, and the diffusion rate of the diffusion marker in the liquid solution is predetermined. In these embodiments, monitoring the time duration that the liquid solution has been deposited on the plate is determined (“deposition duration” hereafter, also the time duration that the plate and the liquid solution are in contact) is to monitor the diffusion of the diffusion maker in the liquid solution.

In some embodiments, the method of monitoring deposition duration during bio/chemical sampling, assays, sensing, and/or processing, comprises the steps of: (a) providing a COF or CROF device that comprises a diffusion marker placed on top of one or both of the plates of the device; (b) depositing the liquid sample on one or both of the plates in the open configuration; (c) bringing the two plates together and compressing the plates into the closed configuration; (d) determining the time duration that the liquid time has been deposited, by monitoring the diffusion of the diffusion marker in the liquid sample.

Using CROF offers the advantages of (a) the CROF makes a sample into a thin film and the diffusion of the diffusion markers is virtually two dimension, and (b) the diffusion time can be images, in many cases, better than that of three dimensional diffusion in a bulk.

In certain embodiments, the sample in a closed configuration is a thin layer of thickness of 1 mm or less. In certain embodiments, the sample in a closed configuration is a thin layer of thickness of about 1 nm, 10 nm, 100 nm, 1 um, 2 um, 3 um, 4 um, 5 um, 10 um, 15 um, 20 um, 25 um, 30 um, 35 um, 40 um, 45 um, 50 um, 75 um, 100 um, 125 um, 150 um, 175 um, 200 um, 300 um, 400 um, 500 um, greater than 500 um, or a range between any two values thereof.

### 3. INTERACTION MARKER AS A CONTACT INDICATOR

Another aspect of the present invention provides the device for monitoring of the time duration that a liquid and a solid are in contact, which, in some embodiments, monitors an interaction of an interaction marker with the liquid, wherein the interaction marker is placed on a location of the solid surface before the liquid contacts the solid surface.

In some embodiments, the device comprises a first plate and an interaction marker. The first plate comprises an inner surface for contacting the sample. Like the diffusion marker discussed above, the interaction marker is positioned on the inner surface of the first plate. The interaction marker is configured to, upon contacting the liquid sample, interact with the sample and such interaction is configured to bring about a signal indicative of the time duration that the sample is in contact with the first plate inner surface.

3.1. Positioning and size of the interaction marker

In some embodiments, the interaction marker is positioned at the center of the first plate inner surface. In some embodiments, the interaction marker is positioned at a predetermined location of the first plate inner surface that is not at the center thereof.

In some embodiments, the sample is deposited directly onto the location of the interaction marker. In some embodiments, the sample is deposited onto a location different from that of the interaction marker, while the sample spreads on the first plate inner surface fast and comes to contact with the interaction marker in a negligible short time.

In some embodiments, the device comprises only one interaction marker.

In some embodiments, there are more than one interaction marker that are positioned at different locations of the first plate inner surface. In some embodiments, the more than one interaction marker is useful for determining the time it takes for the liquid sample to spread on the first plate inner surface.

In some embodiments, the interaction marker has a thickness that is 0.1% or less, 0.5% or less, 1% or less, 2% or less, 5% or less, 10% or less, 20% or less, 25% or less, 30% o or less, 40% or less, or 50% or less of the thickness of the first plate.

In some embodiments, the interaction marker has a thickness that is 10 nm or less, 50 nm or less, 100 nm or less, 1 um or less, 5 um or less, 10 um or less, 20 um or less, 50 um or less, 100 um or less, 500 um or less, 1 mm or less, 2 mm or less, 5 mm or less, 10 mm or less, 50 mm or less, 100 mm or less, 200 mm or less, 500 mm or less, 1 cm or less, 2 cm or less, 5 cm or less, or within a range between any two of these values.

In some embodiments, the interaction marker is a coating on top of the first plate inner surface that is dried thereon.

In some embodiments, the interaction marker is attached to first plate inner surface even upon contacting the sample. In some embodiments, the interaction marker is attached to the first plate inner surface but detachable therefrom upon contacting the sample. In some embodiments, the interaction marker is separate from the first plate inner surface but rests thereon due to gravity or other forces.

In some embodiments, the interaction marker has a lateral area that is 1% or less, 2% or less, 5% or less, 10% or less, 20% or less, 25% or less, 30% or less, 40% or less, 50% or less, 60% or less, 70% or less, 80% or less, 90% or less, 95% or less, 99% or less, or 100% of the lateral area of the first plate inner surface.

### 3.2. Properties of Interaction Signal

In some embodiments, the interaction signal brought about by the interaction between the interaction marker and the sample is a signal selected from a group including, but not limited to, a luminescence signal, a chromatic signal, an electric signal, a magnetic signal, any other forms of signal, and any combination thereof.

As discussed above, the interaction signal is configured to indicate the time duration that the sample is in contact with the first plate inner surface (contact duration). It is thus required that at least one parameter of the interaction signal changes as a predetermined function of the contact duration. In some embodiments, the strength of the interaction signal is correlated with the contact duration. In some cases, when the interaction marker is in contact with the sample, the interaction signal strengthens over the time, while in other cases, the interaction signal weakens over the time. For instance, if the interaction marker is a dye and changes color upon contacting the sample, the color intensity may increase over the time that the sample is in contact with the dye, thereby indicating the contact duration.

In some embodiments, other parameter(s) than the strength of the interaction signal change(s) over on the contact duration. For instance, if the interaction marker is a fluorescent or colorimetric dye and provides fluorescence or visible color change upon contacting the sample, the wavelength of the light it emits or absorbs may change over the time, thereby indicating the contact duration.

In some embodiments, the predetermined function of the at least one parameter of the interaction signal versus the time duration that the interaction marker is in contact with the sample is continuous. In some embodiments, the function is linear, exponential, or in any other continuous form, or any combination thereof.

In some embodiments, the predetermined function of the at least one parameter of the interaction signal versus the time duration that the interaction marker is in contact with the sample is discontinuous, for instance, it could be stepwise. In these embodiments, it is likely that only a reasonable range of contact duration can be determined due to the unstrict correlation between the interaction signal and the contact duration.

In some embodiments, the interaction signal is provided by the interaction marker. For instance, the interaction marker may turn its color or emit fluorescence. In some embodiments, the interaction signal is provided by the deposited sample. For instance, the sample may turn its color upon contacting the interaction marker because of the interaction between the interaction marker and one of the components of the sample. In some embodiments, the interaction signal is provided by a third entity, upon which the interaction between the interaction marker and the sample effects. For instance, an electrical circuitry may be provided to measure the capacitance/conductance change across two different locations of the interaction marker (e.g., an electrical conductor), and the deposition of the sample upon the interaction marker would change the measured capacitance/conductance, thereby leading to a change in the signal provided by the electrical circuitry (e.g., multimeter readout or electric bulb as a part of the circuitry).

### 4. USE OF INTERACTION MARKER IN COF AND CROF PROCESS

Another aspect of the present invention provides a COF device and method that use the interaction marker to monitor the contact duration as discussed above. Yet another aspect of the present invention provides a CROF device and method that use the interaction marker to monitor the contact duration as discussed above.

In some embodiments, the COF or CROF device further comprises an interaction marker that is placed on top of either one or both of the plates, and the interaction marker is configured to interact with the sample, bringing about an interaction signal indicative of the contact duration. In these embodiments, monitoring the deposition duration is realized by monitoring the interaction signal.

In some embodiments, the interaction marker is one single entity or made up of entities of the same species.

In some embodiments, the interaction marker is a combination, group, or mixture of a plurality of entities of different species, and the interaction signal is brought about only by the interaction between the sample and all the entities of each different species in the combination, group, or mixture.

In some embodiments, the interaction marker is a combination, group, or mixture of a plurality of entities of different species, and entities of different species are positioned at different locations of the plates of the COF or CROF device. In some cases, they are positioned at different locations on the inner surface of the same plate. Or in other cases, they are located on the inner surfaces of different plates. In the latter cases, the interaction takes place only after the sample contacts all the plates on which the interaction marker is positioned, and therefore the interaction signal is indicative of the time duration that the sample is in contact with all the plates bearing the interaction marker.

In some embodiments, the method of monitoring deposition duration during bio/chemical sampling, assays, sensing, and/or processing, comprises the steps of: (a) providing a COF or CROF device that comprises an interaction marker placed on top of one or both of the plates of the device; (b) depositing the liquid sample on one or both of the plates in the open configuration; (c) bringing the two plates together and compressing the plates into the closed configuration; (d) determining the time duration that the liquid time has been deposited, by monitoring the interaction signal brought about by the interaction between the interaction marker and the deposited liquid sample.

### 5. DIFFUSION AND INTERACTION MARKER MATERIALS

The diffusion markers can many different substances that are capable of diffusing in a sample and of being distinguished in the samples. For example, embodiments of the present disclosure consist of a method of visualizing a liquid biological sample by applying the sample to a substrate (e.g., a first plate or a second plate) having a diffusible marker, allowing the marker to diffuse with the liquid sample. Embodiments can also include visualizing the diffusion pattern of the diffusion marker that corresponds to an area of the substrate occupied by the liquid sample. Examples of the diffusion markers include, but not limited to, color dyes, fluorescence dyes, scattering centers, and nanoparticles. Those skilled in the art will appreciate that there are a wide variety of substances that provide materials useful in the claimed invention as a diffusion marker. Non-fast, water-soluble diffusion markers that will not interfere with subsequent biological analyses are included in some embodiments of the invention. Bromophenol blue ion and carminic acid ion (cochineal) dyes (or molecules such as their sodium or tris salts) are illustrative diffusion markers that may be used for this purpose. Additional diffusion markers include ethidium bromide and aminoacridine, nucleic acid dyes which are well known in the art, see e.g. U.S. Pat. No. 5,599,932. Bromophenol blue is well known in the art and an example of its use in a solid matrix is disclosed in U.S. Pat. No. 5,049,358 to Lau et al. In addition, carminic acid (cochineal) dyes are well known in the art as disclosed in U.S. Pat. No. 5,147,673 to Schul. These diffusion markers are favored because they are non-toxic and non-interfering with most molecular chemistry. Those skilled in the art appreciate that there are a wide variety of diffusion markers that are useful in this invention and that the examples of Bromophenol blue and carminic acid are provided as illustrative embodiments and are not intended to limit the invention in any way. In addition to the diffusion markers described above, additional substances may provide diffusible materials useful in the claimed invention. For example, metal or carbon sol particles may be useful in accordance with the present invention. Preferably, the detectable species may be a metal-containing particle of the sort fully described in U.S. Pat. No. 4,859,612. In accordance with the concepts and principles of the present invention, metal sol particles having a particle size in the range of from about 50 to about 1000 Angstroms. Such metal particles, and in particular gold sol coated with proteins on their surface have already been described by M. Horisberger et al. in Experimentia, 31, pp. 1147-1149, Oct. 15, 1975. Such particles are intensely colored, either orange, red or violet, depending on particle size. The metal sol particles to be used in accordance with the present invention may be prepared by methodology which is known. For instance, the preparation of gold sol particles is disclosed in an article by G. Frens, Nature, 241, 20-22 (1973). Additionally, the metal sol particles may be metal or metal compounds or polymer nuclei coated with metals or metal compounds, all as described in U.S. Pat. No. 4,313,734. In certain embodiments, an optical label can be used as a diffusion marker and some example of the optical labels have been described in the disclosure.

### 6. MEASUREMENTS OF THE TIME PERIOD OF DIFFUSION AND/OR INTERACTION

Another aspect of the present invention provides the devices, systems, and methods to measure the diffusion and/or interaction time. In certain embodiments, the devices of measuring the diffusion time comprises an imager.

In many applications, the assay testing are not under an ideal/perfect conditions. In certain embodiments of the present invention, for getting accurate diffusion and/or interaction time, artificial intelligence and/or machine learning are used.

In certain embodiments of the present invention, the images taken during an assay operation and/or the samples measured by an assay are analyzed by artificial intelligence and machine learning. The samples include, but not limited to, medical samples, biology samples, environmental samples and chemistry samples.

In certain embodiments of the present invention, the sample is held by a QMAX device. The QMAX device together with imaging plus artificial intelligence and/or machine learning can overcome certain limitations in prior arts.

One important aspect of the present invention is to provide a machine learning framework to enhance the functionality, application scope and the accuracy in assaying using QMAX device, especially when a computer program is used.

In certain embodiments of the present invention, a device and a method for assaying sample and/or assay operation (e.g. diffusion maker's diffusion time) that utilizes QMAX together with imaging plus a machine learning and/or artificial intelligence comprises:

(1) using a QMAX device that has an auxiliary structure in the form of pillars to precisely control the distribution and volume of the sample in assaying, wherein the sample for assaying is loaded into the QMAX device and is kept between the two parallel plates on the QMAX device with an upper plate being transparent for imaging by an imager;

(2) the gap between the two parallel plates in the QMAX device is spaced narrowly—with the distance of the gap being proportional to the size of the analytes to be assayed—by which the analytes in the sample form a single layer between the said plates that can be imaged by an imager on the QMAX device;

(3) the sample volume corresponding to the Aol (area-of-interest) on the upper plate of the QMAX device can be precisely characterized by Aol and the gap—because of the uniformity of the gap between the plates in the QMAX device;

(4) the image on the sample for assaying sandwiched between the Aol x gap in the QMAX device is a pseudo-2D image, because it has the appearance of a 2D image, but it is an image of a 3D sample with its depth being known priori or characterized through other means;

(5) the captured pseudo-2D sample image taken over the Aol of the QMAX device can characterize the location of the analytes, color, shape, counts, and concentration of the analytes in the sample for assaying;

(6) based on abovementioned properties, the captured pseudo-2D image of QMAX device for assaying is amendable to a machine learning framework that applies to analyte detection, localization, identification, segmentation, counting, etc. for assaying in various applications; or

(7) any combination of thereof.

In certain embodiments of the present invention, a machine learning framework for QMAX based devices are implemented into a device that is capable of running an algorithms such as deep learning to discriminatively locate, identify, segment and count analytes (e.g. blood cells) based on the pseudo-2D image captured by the QMAX imager.

In certain embodiments of the present invention, the machine learning improves the images captured by the imager on the QMAX device and reduces the effects of noise and artifacts—including and not limited to air bobbles, dusts, shadows, and pillars.

In certain embodiments of the present invention, the training of machine learning uses the spacers of the QMAX card to reduce the data size of training set.

### 7. EXAMPLES

We tested experimentally an exemplary CROF device for monitoring the deposition duration. The experiment demonstrated the feasibility and validity of using an interaction marker to monitor the deposition duration.

In this experiment, the CROF device comprises an X-plate and a PS substrate plate. The term “X-plate” as used herein refers to a planar plate that has the spacers fixed on the sample surface of the plate. Here the X-plate was made of PMMA and has spacers with a height of 30 um. A mixture of glucose assay reagents (50 unit/ml glucose oxidase, 50 unit/ml horseradish peroxidase, 40 mM 4AAP, and 40 mM TOOS) was nano-printed and dried on the inner surface of the PS substrate plate. At the beginning of the experiment, a liquid sample, which was 3 uL human saliva with 20 mM spike-in glucose, was deposited on the inner surface of the PS substrate plate. Immediately after the sample deposition, the X-plate was brought to cover the substrate plate and hand-pressed against it for less than 1 sec to enter the closed configuration. The plates were then released and remained “self-held” at the closed configuration for the following multiple times of imaging procedure (photos shown below). The mixture of the glucose assay reagents was used here as an interaction marker that reacted with the glucose in the sample and turned into a purple dye.

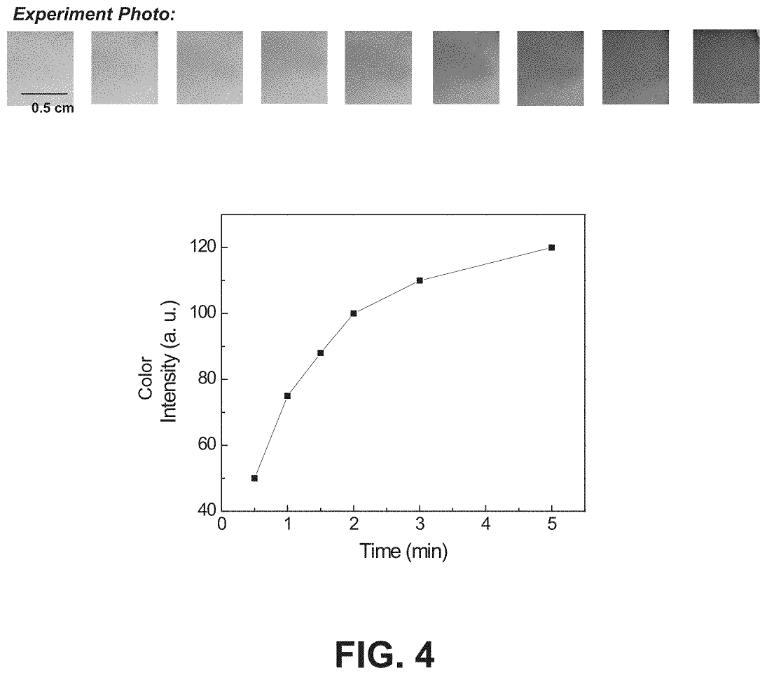

FIG. 4 shows the experimental results using this exemplary device and method. The photos at the top row shows, from left to right, the purple color development over the time after the two plates were hand-pressed to enter the closed configuration (0 min time point), whereas the plot at the bottom summarizes the relationship of the color intensity versus the deposition duration. Considering the transient delay between the sample deposition and the completion of hand-pressing, the deposition duration here was determined as the time difference between the measuring time point and the time point of 0 min. As clearly demonstrated by the photo and the plot, the purple color became darker and darker over the time and was a continuous function of the deposition duration.

Therefore, the concept of using interaction marker to monitor the deposition duration has been empirically validated. In a similar assay setup, based on the experimentally obtained curve as shown in FIG. 4, the deposition duration could be determined by measuring the color intensity of the interaction marker.

### Examples of Present Invention

In certain embodiments of the present disclosure, a device for analyzing a liquid sample can comprise a first plate. In certain embodiments of the present disclosure, a device for analyzing a liquid sample can comprise a diffusion marker. In certain embodiments, the first plate has a sample contact area on its inner surface for contacting the sample. In certain embodiments, the diffusion marker is positioned in the sample contact area of the first plate and is configured to, upon contacting the sample, diffuse in the sample with a pre-determined diffusion rate. In certain embodiments, the diffusion marker is distinguishable from the sample when diffusing in the sample. In certain embodiments, the diffusion of the diffusion marker indicates a time duration that the sample is in contact with the first plate inner surface.

In certain embodiments of the present disclosure, a device for analyzing a liquid sample can comprise a first plate, a second plate, spacers, and a diffusion marker. In certain embodiments, the plates are movable relative to each other into different configurations; one or both plates are flexible. In certain embodiments, both plates have, on its respective inner surface, a sample contact area for contacting a sample. In certain embodiments, the spacers are fixed to the respective inner surface of one or both of the plates and have a predetermined substantially uniform height. In certain embodiments, the diffusion marker is positioned in the sample contact area of one or both of the plates and is configured to, upon contacting the sample, diffuse in the sample with a pre-determined diffusion rate. In certain embodiments, the diffusion marker is distinguishable from the sample when diffusing in the sample. In certain embodiments, one of the configurations is an open configuration, in which: the two plates are partially or entirely separated apart, the spacing between the plates is not regulated by the spacers, and the sample is deposited on one or both of the plates. In certain embodiments, another of the configurations is a closed configuration, which is configured after the sample deposition in the open configuration, and in the closed configuration: at least part of the deposited sample is compressed by the two plates into a layer of uniform thickness that is confined by the two plates, and the uniform thickness of the layer is regulated by the plates and the spacers. In certain embodiments, the diffusion of the diffusion marker indicates a time duration that the sample is in contact with the respective inner surface of the plate on which the diffusion marker is positioned.

In certain embodiments, the diffusion marker is one single entity and diffuses in one direction in the sample, and wherein a diffusion distance of the diffusion marker indicates the time duration that the sample is in contact with the respective plate on which the diffusion marker is positioned. In certain embodiments, the diffusion marker comprises a plurality of entities that all diffuse in one direction in the sample, and wherein a diffusion distance of the diffusion marker indicates the time duration that the sample is in contact with the respective plate on which the diffusion marker is positioned. In certain embodiments, the diffusion marker comprises a plurality of entities, and the diffusion marker as a whole diffuses in more than one direction in the sample, and wherein at least one dimension of the diffusion marker indicates the time duration that the sample is in contact with the respective plate on which the diffusion marker is positioned. In certain embodiments, the diffusion marker diffuses isotropically in the more than one diffusion direction in the liquid solution. In certain embodiments, the diffusion marker diffuses anisotropically in the more than one diffusion direction in the liquid solution. In certain embodiments, the diffusion marker is confined by a physical barrier thereby preventing the diffusion marker from diffusing in one or more directions in the liquid solution. In certain embodiments, the diffusion marker is confined to diffuse in a groove on the inner surface of the plate on which the diffusion marker is positioned. In certain embodiments, the diffusion marker, when diffusing in the sample, is distinguishable from the sample by at least one parameter of the diffusion marker that is selected from the group consisting of light absorption, reflection, transmission, diffraction, scattering, and diffusion; luminescence, heat, viscosity, and magnetism. In certain embodiments, the predetermined diffusion rate is constant. In certain embodiments, the predetermined diffusion rate is a predetermined function of time.

In certain embodiments of the present disclosure, a method of analyzing a liquid sample can comprise obtaining a device of any prior claim. In certain embodiments of the present disclosure, a method of analyzing a liquid sample can comprise depositing the liquid sample on one or both of the plates of the device at the open configuration. In certain embodiments of the present disclosure, a method of analyzing a liquid sample can comprise bringing the two plates together and compressing the plates into the closed configuration. In certain embodiments of the present disclosure, a method of analyzing a liquid sample can comprise, at the closed configuration, analyzing the liquid sample at a time point. In certain embodiments of the present disclosure, a method of analyzing a liquid sample can comprise determining at the time point the time duration that the sample is deposited on one or both of the plates by monitoring the diffusion of the diffusion marker in the deposited sample.

In certain embodiments of the present disclosure, a device for analyzing a liquid sample can comprise a first plate. In certain embodiments of the present disclosure, a device for analyzing a liquid sample can comprise an interaction marker. In certain embodiments, the first plate has a sample contact area on its inner surface for contacting the sample. In certain embodiments, the interaction marker is positioned in the sample contact area of the first plate and is configured to, upon contacting the sample, interact with the sample to bring about an interaction signal. In certain embodiments, the interaction signal is configured to indicate the time duration that the sample is in contact with the first plate inner surface.

In certain embodiments of the present disclosure, a device for analyzing a liquid sample can comprise a first plate. In certain embodiments of the present disclosure, a device for analyzing a liquid sample can comprise a second plate. In certain embodiments of the present disclosure, a device for analyzing a liquid sample can comprise spacers. In certain embodiments of the present disclosure, a device for analyzing a liquid sample can comprise an interaction marker. In certain embodiments, the plates are movable relative to each other into different configurations. In certain embodiments, one or both plates are flexible. In certain embodiments, both plates have, on its respective inner surface, a sample contact area for contacting a sample. In certain embodiments, the spacers are fixed to the respective inner surface of one or both of the plates and have a predetermined substantially uniform height. In certain embodiments, the interaction marker is positioned in the sample contact area of one or both of the plates and is configured to, upon contacting the sample, interact with the sample to bring about an interaction signal. In certain embodiments, one of the configurations is an open configuration, in which: the two plates are partially or entirely separated apart, the spacing between the plates is not regulated by the spacers, and the sample is deposited on one or both of the plates. In certain embodiments, another of the configurations is a closed configuration, which is configured after the sample deposition in the open configuration, and in the closed configuration: at least part of the deposited sample is compressed by the two plates into a layer of uniform thickness that is confined by the two plates, and the uniform thickness of the layer is regulated by the plates and the spacers. In certain embodiments, the interaction signal is configured to indicate the time duration that the sample is in contact with the respective inner surface of the plate on which the interaction marker is positioned.

In certain embodiments, the interaction marker comprises of a plurality of entities of one species. In certain embodiments, the interaction marker comprises of a plurality of entities of different species, and the entities of different species are located on the inner surfaces of different plates. In certain embodiments, the interaction signal is provided by the interaction marker. In certain embodiments, the interaction signal is provided by the sample. In certain embodiments, the interaction signal is provided by an external entity. In certain embodiments, the interaction signal is in a signal form selected from the group consisting of: chromatic signal, luminescence signal, heat, magnetic signal, electric signal, sound, any other electromagnetic signals, and any combination thereof.

In certain embodiments of the present disclosure, a method of analyzing a liquid sample can comprise obtaining any device of the present disclosure. In certain embodiments of the present disclosure, a method of analyzing a liquid sample can comprise depositing the liquid sample on one or both of the plates of the device at the open configuration. In certain embodiments of the present disclosure, a method of analyzing a liquid sample can comprise bringing the two plates together and compressing the plates into the closed configuration. In certain embodiments of the present disclosure, a method of analyzing a liquid sample can comprise, at the closed configuration, analyzing the liquid sample at a time point. In certain embodiments of the present disclosure, a method of analyzing a liquid sample can comprise determining at the time point the time duration that the sample is deposited on one or both of the plates by monitoring the interaction signal.

### Device and Assay With Hicih Uniformity

### Sample Viscosity

In the present invention, the samples to be manipulated and/or analyzed can have a various range of viscosities. For examples, the typical viscosity range is 1.31 to 0.28 (mPa s) from 10 to 100 oC for water; 1.05 to 0.70 (mPa s) from 19 to 37 oC for PBS buffer; 2.4 to 1.45 820 (mPa s) from 17 to 45 oC for plasma; 2.87 to 2.35 (mPa s) from 35 to 42 oC for whole blood; and 0.797 to 0.227 (mPa s) from 0 to 100 oC for methanol. In some embodiments, the sample has a viscosity from 0.1 to 4 (mPa s). In some embodiments, the sample has viscosity of from 4 to 50 (mPa s). In a preferred embodiment, the sample has viscosity of from 0.5 to 3.5 (mPa s).

### Flat Top of Pillar Spacers

In certain embodiments of the present invention, the spacers are pillars that have a flat top and a foot fixed on one plate, wherein the flat top has a smoothness with a small surface variation, and the variation is less than 5, 10 nm, 20 nm, 30 nm, 50 nm, 100 nm, 200 nm, 300 nm, 400 nm, 500 nm, 600 nm, 700 nm, 800 nm, 1000 nm, or in a range between any two of the values. A preferred flat pillar top smoothness is that surface variation of 50 nm or less.

Furthermore, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

### Sidewall Angle of Pillar Spacers

In certain embodiments of the present invention, the spacers are pillars that have a sidewall angle. In some embodiments, the sidewall angle is less than 5 degree (measured from the normal of a surface), 10 degree, 20 degree, 30 degree, 40 degree, 50 degree, 70 degree, or in a range between any two of the values. In a preferred embodiment, the sidewall angle is less 5 degree, 10 degree, or 20 degree.

### Formation of Uniform Thin Fluidic Layer by an Imprecise Force Pressing

In certain embodiment of the present invention, a uniform thin fluidic sample layer is formed by using a pressing with an imprecise force. The term “imprecise pressing force” without adding the details and then adding a definition for imprecise pressing force. As used herein, the term “imprecise” in the context of a force (e.g. “imprecise pressing force”) refers to a force that

(a) has a magnitude that is not precisely known or precisely predictable at the time the force is applied; (b) has a pressure in the range of 0.01 kg/cm² (centimeter square) to 100 kg/cm², (c) varies in magnitude from one application of the force to the next; and (d) the imprecision (i.e. the variation) of the force in (a) and (c) is at least 20% of the total force that actually is applied.

An imprecise force can be applied by human hand, for example, e.g., by pinching an object together between a thumb and index finger, or by pinching and rubbing an object together between a thumb and index finger.

In some embodiments, the imprecise force by the hand pressing has a pressure of 0.01 kg/cm2, 0.1 kg/cm2, 0.5 kg/cm2, 1 kg/cm2, 2 kg/cm2, kg/cm2, 5 kg/cm2, 10 kg/cm2, 20 kg/cm2, 30 kg/cm2, 40 kg/cm2, 50 kg/cm2, 60 kg/cm2, 100 kg/cm2, 150 kg/cm2, 200 kg/cm2, or a range between any two of the values; and a preferred range of 0.1 kg/cm2 to 0.5 kg/cm2, 0.5 kg/cm2 to 1 kg/cm2, 1 kg/cm2 to 5 kg/cm2, 5 kg/cm2 to 10 kg/cm2 (Pressure).

### Spacer Filling Factor.

The term “spacer filling factor” or “filling factor” refers to the ratio of the spacer contact area to the total plate area”, wherein the spacer contact area refers, at a closed configuration, the contact area that the spacer's top surface contacts to the inner surface of a plate, and the total plate area refers the total area of the inner surface of the plate that the flat top of the spacers contact. Since there are two plates and each spacer has two contact surfaces each contacting one plate, the filling fact is the filling factor of the smallest.

For example, if the spacers are pillars with a flat top of a square shape (10 um×10 um), a nearly uniform cross-section and 2 um tall, and the spacers are periodic with a period of 100 um, then the filing factor of the spacer is 1%. If in the above example, the foot of the pillar spacer is a square shape of 15 um×15 um, then the filling factor is still 1% by the definition.

In certain embodiments of the present disclosure, a device for forming a thin fluidic sample layer with a uniform predetermined thickness by pressing can comprise a first plate. In certain embodiments of the present disclosure, a device for forming a thin fluidic sample layer with a uniform predetermined thickness by pressing can comprise a second plate. In certain embodiments of the present disclosure, a device for forming a thin fluidic sample layer with a uniform predetermined thickness by pressing can comprise spacers. In certain embodiments, the plates are movable relative to each other into different configurations. In certain embodiments, one or both plates are flexible. In certain embodiments, each of the plates comprises an inner surface that has a sample contact area for contacting a fluidic sample. In certain embodiments, each of the plates comprises, on its respective outer surface, a force area for applying a pressing force that forces the plates together. In certain embodiments, one or both of the plates comprise the spacers that are permanently fixed on the inner surface of a respective plate. In certain embodiments, the spacers have a predetermined substantially uniform height that is equal to or less than 200 microns, and a predetermined fixed inter-spacer-distance. In certain embodiments, the fourth power of the inter-spacer-distance (ISD) divided by the thickness (h) and the Young's modulus (E) of the flexible plate (ISD⁴/(hE)) is 5×10⁶ um³/GPa or less. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample is deposited in the open configuration and the plates are forced to the closed configuration by applying the pressing force on the force area; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure, a method of forming a thin fluidic sample layer with a uniform predetermined thickness by pressing can comprise depositing a fluidic sample on one or both of the plates when the plates are configured in an open configuration. In certain embodiments, the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for analyzing a fluidic sample can comprise a first plate. In certain embodiments of the present disclosure, a device for analyzing a fluidic sample can comprise a second plate. In certain embodiments of the present disclosure, a device for analyzing a fluidic sample can comprise spacers. In certain embodiments, the plates are movable relative to each other into different configurations. In certain embodiments, one or both plates are flexible. In certain embodiments, each of the plates has, on its respective inner surface, a sample contact area for contacting a fluidic sample. In certain embodiments, one or both of the plates comprise the spacers and the spacers are fixed on the inner surface of a respective plate. In certain embodiments, the spacers have a predetermined substantially uniform height that is equal to or less than 200 microns, and the inter-spacer-distance is predetermined. In certain embodiments, the Young's modulus of the spacers multiplied by the filling factor of the spacers is at least 2 MPa. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample is deposited in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure, a method of forming a thin fluidic sample layer with a uniform predetermined thickness by pressing can comprise depositing a fluidic sample on one or both of the plates when the plates are configured in an open configuration. In certain embodiments, the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing can comprise forcing the two plates into a closed configuration. In certain embodiments, at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for analyzing a fluidic sample can comprise a first plate. In certain embodiments of the present disclosure, a device for analyzing a fluidic sample can comprise a second plate. In certain embodiments, the plates are movable relative to each other into different configurations. In certain embodiments, one or both plates are flexible. In certain embodiments, each of the plates has, on its respective surface, a sample contact area for contacting a sample that contains an analyte. In certain embodiments, one or both of the plates comprise spacers that are permanently fixed to a plate within a sample contact area, wherein the spacers have a predetermined substantially uniform height and a predetermined fixed inter-spacer distance that is at least about 2 times larger than the size of the analyte, up to 200 um, and wherein at least one of the spacers is inside the sample contact area. In certain embodiments, one of the configurations is an open configuration, in which: the two plates are separated apart, the spacing between the plates is not regulated by the spacers, and the sample is deposited on one or both of the plates. In certain embodiments, another of the configurations is a closed configuration which is configured after the sample deposition in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments of the present disclosure, a method of forming a thin fluidic sample layer with a uniform predetermined thickness by pressing can comprise obtaining a device of the present disclosure. In certain embodiments of the present disclosure a method of forming a thin fluidic sample layer with a uniform predetermined thickness by pressing can comprise depositing a fluidic sample on one or both of the plates; when the plates are configured in an open configuration, wherein the open configuration is a configuration in which the two plates are partially or completely separated apart and the spacing between the plates is not regulated by the spacers. In certain embodiments of the present disclosure a method of forming a thin fluidic sample layer with a uniform predetermined thickness by pressing can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments of the present disclosure, a device for forming a thin fluidic sample layer with a uniform predetermined thickness by pressing can comprise a first plate. In certain embodiments of the present disclosure, a device for forming a thin fluidic sample layer with a uniform predetermined thickness by pressing can comprise a second plate. In certain embodiments of the present disclosure, a device for forming a thin fluidic sample layer with a uniform predetermined thickness by pressing can comprise spacers. In certain embodiments, the plates are movable relative to each other into different configurations. In certain embodiments, one or both plates are flexible. In certain embodiments, each of the plates comprises, on its respective inner surface, a sample contact area for contacting and/or compressing a fluidic sample. In certain embodiments, each of the plates comprises, on its respective outer surface, an area for applying a force that forces the plates together. In certain embodiments, one or both of the plates comprise the spacers that are permanently fixed on the inner surface of a respective plate. In certain embodiments, the spacers have a predetermined substantially uniform height that is equal to or less than 200 microns, a predetermined width, and a predetermined fixed inter-spacer-distance. In certain embodiments, a ratio of the inter-spacer-distance to the spacer width is 1.5 or larger. In certain embodiments, at least one of the spacers is inside the sample contact area. In certain embodiments, one of the configurations is an open configuration, in which: the two plates are partially or completely separated apart, the spacing between the plates is not regulated by the spacers, and the sample is deposited on one or both of the plates. In certain embodiments, another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments of the present disclosure, a method of forming a thin fluidic sample layer with a uniform predetermined thickness by pressing with an imprecise pressing force can comprise obtaining a device of the present disclosure. In certain embodiments of the present disclosure, a method of forming a thin fluidic sample layer with a uniform predetermined thickness by pressing with an imprecise pressing force can comprise obtaining a fluidic sample. In certain embodiments of the present disclosure, a method of forming a thin fluidic sample layer with a uniform predetermined thickness by pressing with an imprecise pressing force can comprise depositing the sample on one or both of the plates; when the plates are configured in an open configuration, wherein the open configuration is a configuration in which the two plates are partially or completely separated apart and the spacing between the plates is not regulated by the spacers. In certain embodiments of the present disclosure, a method of forming a thin fluidic sample layer with a uniform predetermined thickness by pressing with an imprecise pressing force can comprise forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments, the spacers have a shape of pillar with a foot fixed on one of the plates and a flat top surface for contacting the other plate. In certain embodiments, the spacers have a shape of pillar with a foot fixed on one of the plates, a flat top surface for contacting the other plate, substantially uniform cross-section. In certain embodiments, the spacers have a shape of pillar with a foot fixed on one of the plates and a flat top surface for contacting the other plate, wherein the flat top surface of the pillars has a variation in less than 10 nm. In certain embodiments, the spacers have a shape of pillar with a foot fixed on one of the plates and a flat top surface for contacting the other plate, wherein the flat top surface of the pillars has a variation in less than 50 nm. In certain embodiments, the spacers have a shape of pillar with a foot fixed on one of the plates and a flat top surface for contacting the other plate, wherein the flat top surface of the pillars has a variation in less than 50 nm. In certain embodiments, the spacers have a shape of pillar with a foot fixed on one of the plates and a flat top surface for contacting the other plate, wherein the flat top surface of the pillars has a variation in less than 10 nm, 20 nm, 30 nm, 100 nm, 200 nm, or in a range of any two of the values.

In certain embodiments, the Young's modulus of the spacers multiplied by the filling factor of the spacers is at least 2 MPa. In certain embodiments, the sample comprises an analyte and the predetermined constant inter-spacer distance is at least about 2 times larger than the size of the analyte, up to 200 um. In certain embodiments, the sample comprise an analyte, the predetermined constant inter-spacer distance is at least about 2 times larger than the size of the analyte, up to 200 um, and the Young's modulus of the spacers multiplied by the filling factor of the spacers is at least 2 MPa.

In certain embodiments, a fourth power of the inter-spacer-distance (IDS) divided by the thickness (h) and the Young's modulus (E) of the flexible plate (ISD\hat{ }4/(hE)) is 5×10\hat{ }6 um\hat{ }3/GPa or less. In certain embodiments, a fourth power of the inter-spacer-distance (IDS) divided by the thickness and the Young's modulus of the flexible plate (ISD\hat{ }4/(hE)) is 1×10\hat{ }6 um\hat{ }3/GPa or less. In certain embodiments, a fourth power of the inter-spacer-distance (IDS) divided by the thickness and the Young's modulus of the flexible plate (ISD\hat{ }4/(hE)) is 5×10\hat{ }5 um\hat{ }3/GPa or less. In certain embodiments, the Young's modulus of the spacers multiplied by the filling factor of the spacers is at least 2 MPa, and a fourth power of the inter-spacer-distance (IDS) divided by the thickness and the Young's modulus of the flexible plate (ISD\hat{ }4/(hE)) is 1×10\hat{ }5 um\hat{ }3/GPa or less. In certain embodiments, the Young's modulus of the spacers multiplied by the filling factor of the spacers is at least 2 MPa, and a fourth power of the inter-spacer-distance (IDS) divided by the thickness and the Young's modulus of the flexible plate (ISD\hat{ }4/(hE)) is 1×10\hat{ }4 um\hat{ }3/GPa or less. In certain embodiments, the Young's modulus of the spacers multiplied by the filling factor of the spacers is at least 20 MPa.

In certain embodiments of the present disclosure, the ratio of the inter-spacing distance of the spacers to the average width of the spacer is 2 or larger. In certain embodiments, the ratio of the inter-spacing distance of the spacers to the average width of the spacer is 2 or larger, and the Young's modulus of the spacers multiplied by the filling factor of the spacers is at least 2 MPa. In certain embodiments, the inter-spacer distance that is at least about 2 times larger than the size of the analyte, up to 200 um. In certain embodiments, a ratio of the inter-spacer-distance to the spacer width is 1.5 or larger. In certain embodiments, a ratio of the width to the height of the spacer is 1 or larger. In certain embodiments, a ratio of the width to the height of the spacer is 1.5 or larger. In certain embodiments, a ratio of the width to the height of the spacer is 2 or larger. In certain embodiments, a ratio of the width to the height of the spacer is larger than 2, 3, 5, 10, 20, 30, 50, or in a range of any two the value.

In certain embodiments, a force that presses the two plates into the closed configuration is an imprecise pressing force. In certain embodiments, a force that presses the two plates into the closed configuration is an imprecise pressing force provided by human hand. In certain embodiments, the forcing of the two plates to compress at least part of the sample into a layer of substantially uniform thickness comprises a use of a conformable pressing, either in parallel or sequentially, an area of at least one of the plates to press the plates together to a closed configuration, wherein the conformable pressing generates a substantially uniform pressure on the plates over the at least part of the sample, and the pressing spreads the at least part of the sample laterally between the sample contact surfaces of the plates, and wherein the closed configuration is a configuration in which the spacing between the plates in the layer of uniform thickness region is regulated by the spacers; and wherein the reduced thickness of the sample reduces the time for mixing the reagents on the storage site with the sample. In certain embodiments, the pressing force is an imprecise force that has a magnitude which is, at the time that the force is applied, either (a) unknown and unpredictable, or (b) cannot be known and cannot be predicted within an accuracy equal or better than 20% of the average pressing force applied. In certain embodiments, the pressing force is an imprecise force that has a magnitude which is, at the time that the force is applied, either (a) unknown and unpredictable, or (b) cannot be known and cannot be predicted within an accuracy equal or better than 30% of the average pressing force applied. In certain embodiments, the pressing force is an imprecise force that has a magnitude which is, at the time that the force is applied, either (a) unknown and unpredictable, or (b) cannot be known and cannot be predicted within an accuracy equal or better than 30% of the average pressing force applied; and wherein the layer of highly uniform thickness has a variation in thickness uniform of 20% or less. In certain embodiments, the pressing force is an imprecise force that has a magnitude which cannot, at the time that the force is applied, be determined within an accuracy equal or better than 30%, 40%, 50%, 70%, 100%, 200%, 300%, 500%, 1000%, 2000%, or in a range between any of the two values.

In certain embodiments of the present disclosure, the flexible plate has a thickness of in the range of 10 um to 200 um. In certain embodiments, the flexible plate has a thickness of in the range of 20 um to 100 um. In certain embodiments, the flexible plate has a thickness of in the range of 25 um to 180 um. In certain embodiments, the flexible plate has a thickness of in the range of 200 um to 260 um. In certain embodiments, the flexible plate has a thickness of equal to or less than 250 um, 225 um, 200 um, 175 um, 150 um, 125 um, 100 um, 75 um, 50 1100 um, 25 um, 10 um, 5 um, 1 um, or in a range between the two of the values. In certain embodiments, the sample has a viscosity in the range of 0.1 to 4 (mPa s). In certain embodiments, the flexible plate has a thickness of in the range of 200 um to 260 um. In certain embodiments, the flexible plate has a thickness in the range of 20 um to 200 um and Young's modulus in the range 0.1 to 5 GPa.

In certain embodiments of the present disclosure, the sample deposition is a deposition directly from a subject to the plate without using any transferring devices. In certain embodiments, during the deposition, the amount of the sample deposited on the plate is unknown. In certain embodiments, the method further comprises an analyzing that analyze the sample. In certain embodiments, the analyzing comprises calculating the volume of a relevant sample volume by measuring the lateral area of the relevant sample volume and calculating the volume from the lateral area and the predetermined spacer height. In certain embodiments, the analyzing step (e) comprises measuring: i. imaging, ii. luminescence selected from photoluminescence, electroluminescence, and electrochemiluminescence, iii. surface Raman scattering, iv. electrical impedance selected from resistance, capacitance, and inductance, or v. any combination of i-iv. In certain embodiments, the analyzing comprises reading, image analysis, or counting of the analyte, or a combination of thereof. In certain embodiments, the sample contains one or plurality of analytes, and one or both plate sample contact surfaces comprise one or a plurality of binding sites that each binds and immobilize a respective analyte. In certain embodiments, one or both plate sample contact surfaces comprise one or a plurality of storage sites that each stores a reagent or reagents, wherein the reagent(s) dissolve and diffuse in the sample. In certain embodiments, one or both plate sample contact surfaces comprises one or a plurality of amplification sites that are each capable of amplifying a signal from the analyte or a label of the analyte when the analyte or label is within 500 nm from an amplification site. In certain embodiments, i. one or both plate sample contact surfaces comprise one or a plurality of binding sites that each binds and immobilize a respective analyte; or ii. one or both plate sample contact surfaces comprise, one or a plurality of storage sites that each stores a reagent or reagents; wherein the reagent(s) dissolve and diffuse in the sample, and wherein the sample contains one or plurality of analytes; or iii. one or a plurality of amplification sites that are each capable of amplifying a signal from the analyte or a label of the analyte when the analyte or label is 500 nm from the amplification site; or iv. any combination of i to iii.

In certain embodiments, the liquid sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

In certain embodiments, the layer of uniform thickness in the closed configuration is less than 150 um. In certain embodiments, the pressing is provided by a pressured liquid, a pressed gas, or a conformal material. In certain embodiments, the analyzing comprises counting cells in the layer of uniform thickness. In certain embodiments, the analyzing comprises performing an assay in the layer of uniform thickness. In certain embodiments, In certain embodiments, the assay is a binding assay or biochemical assay. In certain embodiments, the sample deposited has a total volume less 0.5 uL. In certain embodiments, multiple drops of sample are deposited onto one or both of the plates.

In certain embodiments, the inter-spacer distance is in the range of 1 μm to 120 μm. In certain embodiments, the inter-spacer distance is in the range of 120 μm to 50 μm. In certain embodiments, the inter-spacer distance is in the range of 120 μm to 200 μm. In certain embodiments, the flexible plates have a thickness in the range of 20 um to 250 um and Young's modulus in the range 0.1 to 5 GPa. In certain embodiments, for a flexible plate, the thickness of the flexible plate times the Young's modulus of the flexible plate is in the range 60 to 750 GPa-um.

In certain embodiments, the layer of uniform thickness sample is uniform over a lateral area that is at least 1 mm². In certain embodiments, the layer of uniform thickness sample is uniform over a lateral area that is at least 3 mm². In certain embodiments, the layer of uniform thickness sample is uniform over a lateral area that is at least 5 mm². In certain embodiments, In certain embodiments, the layer of uniform thickness sample is uniform over a lateral area that is at least 10 mm². In certain embodiments, the layer of uniform thickness sample is uniform over a lateral area that is at least 20 mm². In certain embodiments, the layer of uniform thickness sample is uniform over a lateral area that is in a range of 20 mm² to 100 mm². In certain embodiments, the layer of uniform thickness sample has a thickness uniformity of up to +/−5% or better. In certain embodiments, the layer of uniform thickness sample has a thickness uniformity of up to +1-10% or better. In certain embodiments, the layer of uniform thickness sample has a thickness uniformity of up to +/−20% or better. In certain embodiments, the layer of uniform thickness sample has a thickness uniformity of up to +/−30% or better. In certain embodiments, the layer of uniform thickness sample has a thickness uniformity of up to +/−40% or better. In certain embodiments, the layer of uniform thickness sample has a thickness uniformity of up to +/−50% or better.

In certain embodiments, the spacers are pillars with a cross-sectional shape selected from round, polygonal, circular, square, rectangular, oval, elliptical, or any combination of the same. In certain embodiments, the spacers have pillar shape, have a substantially flat top surface, and have substantially uniform cross-section, wherein, for each spacer, the ratio of the lateral dimension of the spacer to its height is at least 1. In certain embodiments, the inter spacer distance is periodic. In certain embodiments, the spacers have a filling factor of 1% or higher, wherein the filling factor is the ratio of the spacer contact area to the total plate area. In certain embodiments, the Young's modulus of the spacers times the filling factor of the spacers is equal or larger than 20 MPa, wherein the filling factor is the ratio of the spacer contact area to the total plate area. In certain embodiments, the spacing between the two plates at the closed configuration is in less 200 um. In certain embodiments, the spacing between the two plates at the closed configuration is a value selected from between 1.8 um and 3.5 um. In certain embodiments, the spacing are fixed on a plate by directly embossing the plate or injection molding of the plate. In certain embodiments, the materials of the plate and the spacers are selected from polystyrene, PMMA, PC, COC, COP, or another plastic. In certain embodiments, the spacers have a pillar shape, and the sidewall corners of the spacers have a round shape with a radius of curvature at least 1 μm. In certain embodiments, the spacers have a density of at least 1000/mm². In certain embodiments, at least one of the plates is transparent. In certain embodiments, the mold used to make the spacers is fabricated by a mold containing features that are fabricated by either (a) directly reactive ion etching or ion beam etched or (b) by a duplication or multiple duplication of the features that are reactive ion etched or ion beam 1190 etched.

In certain embodiments, the spacers are configured, such that the filling factor is in the range of 1% to 5%. In certain embodiments, the surface variation is relative to the spacer height and the ratio of the pillar flat top surface variation to the spacer height is less than 0.5%, 1%, 3%, 5%, 7%, 10%, 15%, 20%, 30%,40%, or in a range between any two of the values. A preferred flat pillar top smoothness has a ratio of the pillar flat top surface variation to the spacer height is less than 2%, 5%, or 10%. In certain embodiments, the spacers are configured, such that the filling factor is in the range of 1% to 5%. In certain embodiments, the spacers are configured, such that the filling factor is in the range of 5% to 10%. In certain embodiments, the spacers are configured, such that the filling factor is in the range of 10% to 20%. In certain embodiments, the spacers are configured, such that the filling factor is in the range of 20% to 30%. In certain embodiments, the spacers are configured, such that the filling factor is 5%, 10%, 20%, 30%, 40%,50%, or in a range of any two of the values. In certain embodiments, the spacers are configured, such that the filling factor is 50%, 60%, 70%, 80%, or in a range of any two of the values.

In certain embodiments, the spacers are configured, such that the filling factor multiplies the Young's modulus of the spacer is in the range of 2 MPa and 10 MPa. In certain embodiments, the spacers are configured, such that the filling factor multiplies the Young's modulus of the spacer is in the range of 10 MPa and 20 MPa. In certain embodiments, the spacers are configured, such that the filling factor multiplies the Young's modulus of the spacer is in the range of 20 MPa and 40 MPa. In certain embodiments, the spacers are configured, such that the filling factor multiplies the Young's modulus of the spacer is in the range of 40 MPa and 80 MPa. In certain embodiments, the spacers are configured, such that the filling factor multiplies the Young's modulus of the spacer is in the range of 80 MPa and 120 MPa. In certain embodiments, the spacers are configured, such that the filling factor multiplies the Young's modulus of the spacer is in the range of 120 MPa to 150 MPa.

In certain embodiments, the device further comprises a dry reagent coated on one or both plates. In certain embodiments, the device further comprises, on one or both plates, a dry binding site that has a predetermined area, wherein the dry binding site binds to and immobilizes an analyte in the sample. In certain embodiments, the device further comprises, on one or both plates, a releasable dry reagent and a release time control material that delays the time that the releasable dry regent is released into the sample. In certain embodiments, the release time control material delays the time that the dry regent starts is released into the sample by at least 3 seconds. In certain embodiments, the regent comprises anticoagulant and/or staining reagent(s). In certain embodiments, the reagent comprises cell lysing reagent(s). In certain embodiments, the device further comprises, on one or both plates, one or a plurality of dry binding sites and/or one or a plurality of reagent sites. In certain embodiments, the analyte comprises a molecule (e.g., a protein, peptides, DNA, RNA, nucleic acid, or other molecule), cells, tissues, viruses, and nanoparticles with different shapes. In certain embodiments, the analyte comprises white blood cells, red blood cells and platelets. In certain embodiments, the analyte is stained.

In certain embodiments, the spacers regulating the layer of uniform thickness have a filling factor of at least 1%, wherein the filling factor is the ratio of the spacer area in contact with the layer of uniform thickness to the total plate area in contact with the layer of uniform thickness. In certain embodiments, for spacers regulating the layer of uniform thickness, the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness. In certain embodiments, for a flexible plate, the thickness of the flexible plate times the Young's modulus of the flexible plate is in the range 60 to 750 GPa-um. In certain embodiments, for a flexible plate, the fourth power of the inter-spacer-distance (ISD) divided by the thickness of the flexible plate (h) and the Young's modulus (E) of the flexible plate, ISD⁴/(hE), is equal to or less than 10⁶ um³/GPa.

In certain embodiments, one or both plates comprises a location marker, either on a surface of or inside the plate, that provide information of a location of the plate. In certain embodiments, one or both plates comprises a scale marker, either on a surface of or inside the plate, that provide information of a lateral dimension of a structure of the sample and/or the plate. In certain embodiments, one or both plates comprises an imaging marker, either on surface of or inside the plate, that assists an imaging of the sample. In certain embodiments, the spacers functions as a location marker, a scale marker, an imaging marker, or any combination of thereof.

In certain embodiments, the average thickness of the layer of uniform thickness is about equal to a minimum dimension of an analyte in the sample. In certain embodiments, the inter-spacer distance is in the range of 7 μm to 50 μm. In certain embodiments, the inter-spacer distance is in the range of 50 μm to 120 μm. In certain embodiments, the inter-spacer distance is in the range of 120 μm to 200 μm (micron). In certain embodiments, the inter-spacer distance is substantially periodic. In certain embodiments, the spacers are pillars with a cross-sectional shape selected from round, polygonal, circular, square, rectangular, oval, elliptical, or any combination of the same.

In certain embodiments, the spacers have a pillar shape and have a substantially flat top surface, wherein, for each spacer, the ratio of the lateral dimension of the spacer to its height is at least 1. In certain embodiments, each spacer has the ratio of the lateral dimension of the spacer to its height is at least 1. In certain embodiments, the minimum lateral dimension of spacer is less than or substantially equal to the minimum dimension of an analyte in the sample. In certain embodiments, the minimum lateral dimension of spacer is in the range of 0.5 um to 100 um. In certain embodiments, the minimum lateral dimension of spacer is in the range of 0.5 um to 10 um.

In certain embodiments, the sample is blood. In certain embodiments, the sample is whole blood without dilution by liquid. In certain embodiments, the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine. In certain embodiments, the sample is a biological sample, an environmental sample, a chemical sample, or clinical sample.

In certain embodiments, the spacers have a pillar shape, and the sidewall corners of the spacers have a round shape with a radius of curvature at least 1 μm. In certain embodiments, the spacers have a density of at least 100/mm². In certain embodiments, the spacers have a density of at least 1000/mm². In certain embodiments, at least one of the plates is transparent. In certain embodiments, at least one of the plates is made from a flexible polymer. In certain embodiments, for a pressure that compresses the plates, the spacers are not compressible and/or, independently, only one of the plates is flexible. In certain embodiments, the flexible plate has a thickness in the range of 10 um to 200 um. In certain embodiments, the variation is less than 30%. In certain embodiments, the variation is less than 10%. In certain embodiments, the variation is less than 5%.

In certain embodiments, the first and second plates are connected and are configured to be changed from the open configuration to the closed configuration by folding the plates. In certain embodiments, the first and second plates are connected by a hinge and are configured to be changed from the open configuration to the closed configuration by folding the plates along the hinge. In certain embodiments, the first and second plates are connected by a hinge that is a separate material to the plates, and are configured to be changed from the open configuration to the closed configuration by folding the plates along the hinge. In certain embodiments, the first and second plates are made in a single piece of material and are configured to be changed from the open configuration to the closed configuration by folding the plates. In certain embodiments, the layer of uniform thickness sample is uniform over a lateral area that is at least 1 mm².

In certain embodiments, the device is configured to analyze the sample in 60 seconds or less. In certain embodiments, at the closed configuration, the final sample thickness device is configured to analyze the sample in 60 seconds or less. In certain embodiments, at the closed configuration, the final sample thickness device is configured to analyze the sample in 10 seconds or less.

In certain embodiments, the dry binding site comprises a capture agent. In certain embodiments, the dry binding site comprises an antibody or nucleic acid. In certain embodiments, the releasable dry reagent is a labeled reagent. In certain embodiments, the releasable dry reagent is a fluorescently-labeled reagent. In certain embodiments, the releasable dry reagent is a fluorescently-labeled antibody. In certain embodiments, the releasable dry reagent is a cell stain. In certain embodiments, the releasable dry reagent is a cell lysing.

In certain embodiments, the detector is an optical detector that detects an optical signal. In certain embodiments, the detector is an electric detector that detect electrical signal. In certain embodiments, the spacing are fixed on a plate by directly embossing the plate or injection molding of the plate. In certain embodiments, the materials of the plate and the spacers are selected from polystyrene, PMMA, PC, COC, COP, or another plastic.

In certain embodiments of the present disclosure, a system for rapidly analyzing a sample using a mobile phone can comprise a device of any prior embodiment. In certain embodiments of the present disclosure, a system for rapidly analyzing a sample using a mobile phone can comprise a mobile communication device. In certain embodiments, the mobile communication device can comprise one or a plurality of cameras for the detecting and/or imaging the sample. In certain embodiments, the mobile communication device can comprise electronics, signal processors, hardware and software for receiving and/or processing the detected signal and/or the image of the sample and for remote communication. In certain embodiments, the mobile communication device can comprise a light source from either the mobile communication device or an external source. In same embodiments, the detector in the devices or methods of any prior embodiment is provided by the mobile communication device, and detects an analyte in the sample at the closed configuration.

In certain embodiments, one of the plates has a binding site that binds an analyte, wherein at least part of the uniform sample thickness layer is over the binding site, and is substantially less than the average lateral linear dimension of the binding site. In certain embodiments, any system of the present disclosure can comprise a housing configured to hold the sample and to be mounted to the mobile communication device. In certain embodiments, the housing comprises optics for facilitating the imaging and/or signal processing of the sample by the mobile communication device, and a mount configured to hold the optics on the mobile communication device. In certain embodiments, an element of the optics in the housing is movable relative to the housing. In certain embodiments, the mobile communication device is configured to communicate test results to a medical professional, a medical facility or an insurance company. In certain embodiments, the mobile communication device is further configured to communicate information on the test and the subject with the medical professional, medical facility or insurance company. In certain embodiments, the mobile communication device is further configured to communicate information of the test to a cloud network, and the cloud network process the information to refine the test results. In certain embodiments, the mobile communication device is further configured to communicate information of the test and the subject to a cloud network, the cloud network process the information to refine the test results, and the refined test results will send back the subject. In certain embodiments, the mobile communication device is configured to receive a prescription, diagnosis or a recommendation from a medical professional. In certain embodiments, the mobile communication device is configured with hardware and software to capture an image of the sample. In certain embodiments, the mobile communication device is configured with hardware and software to analyze a test location and a control location in in image. In certain embodiments, the mobile communication device is configured with hardware and software to compare a value obtained from analysis of the test location to a threshold value that characterizes the rapid diagnostic test.

In certain embodiments of the present disclosure, at least one of the plates comprises a storage site in which assay reagents are stored. In certain embodiments, at least one of the cameras reads a signal from the device. In certain embodiments, the mobile communication device communicates with the remote location via a wifi or cellular network. In certain embodiments, the mobile communication device is a mobile phone.

In certain embodiments of the present disclosure, a method for rapidly analyzing an analyte in a sample using a mobile phone can comprise depositing a sample on the device of any prior system embodiment. In certain embodiments of the present disclosure, a method for rapidly analyzing an analyte in a sample using a mobile phone can comprise assaying an analyte in the sample deposited on the device to generate a result. In certain embodiments of the present disclosure, a method for rapidly analyzing an analyte in a sample using a mobile phone can comprise communicating the result from the mobile communication device to a location remote from the mobile communication device.

In certain embodiments, the analyte comprises a molecule (e.g., a protein, peptides, DNA, RNA, nucleic acid, or other molecule), cells, tissues, viruses, and nanoparticles with different shapes. In certain embodiments, the analyte comprises white blood cell, red blood cell and platelets. In certain embodiments, the assaying comprises performing a white blood cells differential assay. In certain embodiments, a method of the present disclosure can comprise analyzing the results at the remote location to provide an analyzed result. In certain embodiments, a method of the present disclosure can comprise communicating the analyzed result from the remote location to the mobile communication device. In certain embodiments, the analysis is done by a medical professional at a remote location. In certain embodiments, the mobile communication device receives a prescription, diagnosis or a recommendation from a medical professional at a remote location.

In certain embodiments, the sample is a bodily fluid. In certain embodiments, the bodily fluid is blood, saliva or urine. In certain embodiments, the sample is whole blood without dilution by a liquid. In certain embodiments, the assaying step comprises detecting an analyte in the sample. In certain embodiments, the analyte is a biomarker. In certain embodiments, the analyte is a protein, nucleic acid, cell, or metabolite. In certain embodiments, the method comprises counting the number of red blood cells. In certain embodiments, the method comprises counting the number of white blood cells. In certain embodiments, the method comprises staining the cells in the sample and counting the number of neutrophils, lymphocytes, monocytes, eosinophils and basophils. In certain embodiments, the assay done in step (b) is a binding assay or a biochemical assay.

In certain embodiments of the present disclosure, a method for analyzing a sample can comprise obtaining a device of any prior device embodiment. In certain embodiments of the present disclosure, a method for analyzing a sample can comprise depositing the sample onto one or both pates of the device. In certain embodiments of the present disclosure, a method for analyzing a sample can comprise placing the plates in a closed configuration and applying an external force over at least part of the plates. In certain embodiments of the present disclosure, a method for analyzing a sample can comprise analyzing the layer of uniform thickness while the plates are the closed configuration.

In certain embodiments, the first plate further comprises, on its surface, a first predetermined assay site and a second predetermined assay site, wherein the distance between the edges of the assay site is substantially larger than the thickness of the uniform thickness layer when the plates are in the closed position, wherein at least a part of the uniform thickness layer is over the predetermined assay sites, and wherein the sample has one or a plurality of analytes that are capable of diffusing in the sample. In certain embodiments, the first plate has, on its surface, at least three analyte assay sites, and the distance between the edges of any two neighboring assay sites is substantially larger than the thickness of the uniform thickness layer when the plates are in the closed position, wherein at least a part of the uniform thickness layer is over the assay sites, and wherein the sample has one or a plurality of analytes that are capable of diffusing in the sample. In certain embodiments, the first plate has, on its surface, at least two neighboring analyte assay sites that are not separated by a distance that is substantially larger than the thickness of the uniform thickness layer when the plates are in the closed position, wherein at least a part of the uniform thickness layer is over the assay sites, and wherein the sample has one or a plurality of analytes that are capable of diffusing in the sample. In certain embodiments, the analyte assay area is between a pair of electrodes. In certain embodiments, the assay area is defined by a patch of dried reagent. In certain embodiments, the assay area binds to and immobilizes the analyte. In certain embodiments, the assay area is defined by a patch of binding reagent that, upon contacting the sample, dissolves into the sample, diffuses in the sample, and binds to the analyte. In certain embodiments, the inter-spacer distance is in the range of 14 μm to 200 μm. In certain embodiments, the inter-spacer distance is in the range of 7 μm to 20 μm. In certain embodiments, the spacers are pillars with a cross-sectional shape selected from round, polygonal, circular, square, rectangular, oval, elliptical, or any combination of the same. In certain embodiments, the spacers have a pillar shape and have a substantially flat top surface, wherein, for each spacer, the ratio of the lateral dimension of the spacer to its height is at least 1. In certain embodiments, the spacers have a pillar shape, and the sidewall corners of the spacers have a round shape with a radius of curvature at least 1 μm. In certain embodiments, the spacers have a density of at least 1000/mm². In certain embodiments, at least one of the plates is transparent. In certain embodiments, at least one of the plates is made from a flexible polymer. In certain embodiments, only one of the plates is flexible. In certain embodiments, the area-determination device is a camera. In certain embodiments, an area in the sample contact area of a plate, wherein the area is less than 1/100, 1/20, 1/10, ⅙, ⅕, ¼, ⅓, ½, ⅔ of the sample contact area, or in a range between any of the two values. In certain embodiments, the area-determination device comprises a camera and an area in the sample contact area of a plate, wherein the area is in contact with the sample.

In certain embodiments, the deformable sample comprises a liquid sample. In certain embodiments, the imprecision force has a variation at least 30% of the total force that actually is applied. In certain embodiments, the imprecision force has a variation at least 20%, 30%, 40%, 50%, 60, 70%, 80%, 90% 100%, 150%, 200%, 300%, 500%, or in a range of any two values, of the total force that actually is applied. In certain embodiments, the spacers have a flat top. In certain embodiments, the device is further configured to have, after the pressing force is removed, a sample thickness that is substantially the same in thickness and uniformity as that when the force is applied. In certain embodiments, the imprecise force is provided by human hand. In certain embodiments, the inter spacer distance is substantially constant. In certain embodiments, the inter spacer distance is substantially periodic in the area of the uniform sample thickness area. In certain embodiments, the multiplication product of the filling factor and the Young's modulus of the spacer is 2 MPa or larger. In certain embodiments, the force is applied by hand directly or indirectly. In certain embodiments, the force applied is in the range of 1 N to 20 N. In certain embodiments, the force applied is in the range of 20 N to 200 N. In certain embodiments, the highly uniform layer has a thickness that varies by less than 15%, 10%, or 5% of an average thickness. In certain embodiments, the imprecise force is applied by pinching the device between a thumb and forefinger. In certain embodiments, the predetermined sample thickness is larger than the spacer height. In certain embodiments, the device holds itself in the closed configuration after the pressing force has been removed. In certain embodiments, the uniform thickness sample layer area is larger than that area upon which the pressing force is applied. In certain embodiments, the spacers do not significantly deform during application of the pressing force. In certain embodiments, the pressing force is not predetermined beforehand and is not measured. In certain embodiments, the fluidic sample is replaced by a deformable sample and the embodiments for making at least a part of the fluidic sample into a uniform thickness layer can make at least a part of the deformable sample into a uniform thickness layer. In certain embodiments, the inter spacer distance is periodic. In certain embodiments, the spacers have a flat top. In certain embodiments, the inter spacer distance is at least two times large than the size of the targeted analyte in the sample.

### Manufacturing of Q-Card

In certain embodiments of the present disclosure, a Q-Card can comprise a first plate. In certain embodiments of the present disclosure, a Q-Card can comprise a second plate. In certain embodiments of the present disclosure, a Q-Card can comprise a hinge. In certain embodiments, the first plate, that is about 200 nm to 1500 nm thick, comprises, on its inner surface, (a) a sample contact area for contacting a sample, and (b) a sample overflow dam that surrounds the sample contact area is configured to present a sample flow outside of the dam. In certain embodiments, the second plate is 10 um to 250 um thick and comprises, on its inner surface, (a) a sample contact area for contacting a sample, and (b) spacers on the sample contact area. In certain embodiments, the hinge that connect the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, (a) a sample contact area for contacting a sample, (b) a sample overflow dam that surrounds the sample contact area is configured to present a sample flow outside of the dam, and (c) spacers on the sample contact area. In certain embodiments, the second plate, that is 10 um to 250 um thick, comprises, on its inner surface, a sample contact area for contacting a sample. In certain embodiments, the hinge connects the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, (a) a sample contact area for contacting a sample, and (b) spacers on the sample contact area. In certain embodiments, the second plate, that is 10 um to 250 um thick, comprises, on its inner surface, (a) a sample contact area for contacting a sample, and (b) a sample overflow dam that surrounds the sample contact area is configured to present a sample flow outside of the dam. In certain embodiments, the hinge connects the first and the second plates. In certain embodiments, the first and second plate are movable relative to each other around the axis of the hinge.

In certain embodiments of the present disclosure, an embodiment of the Q-Card can comprise a first plate. In certain embodiments of the present disclosure, an embodiment of the Q-Card can comprise a second plate. In certain embodiments of the present disclosure, an embodiment of the Q-Card can comprise a hinge. In certain embodiments, the first plate, that is about 200 nm to 1500 nm thick, comprises, on its inner surface, a sample contact area for contacting a sample. In certain embodiments, the second plate, that is 10 um to 250 um thick, comprises, on its inner surface, (a) a sample contact area for contacting a sample, (b) a sample overflow dam that surrounds the sample contact area is configured to present a sample flow outside of the dam, and (c) spacers on the sample contact area. In certain embodiments, the hinge connects the first and the second plates. In certain embodiments, the first and second plate are movable relative to each other around the axis of the hinge.

In certain embodiments of the present disclosure, a method for fabricating any Q-Card of the present disclosure can comprise injection molding of the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise Laser cutting the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise injection molding and laser cutting the first plate. In certain embodiments of the present disclosure, a method for fabricating any Q-Card of the present disclosure can comprise nanoimprinting or extrusion printing of the second plate.

In certain embodiments of the present disclosure, a method for fabricating any Q-Card of the present disclosure can comprise nanoimprinting or extrusion printing to fabricated both the first and the second plate.

In certain embodiments of the present disclosure, a method for fabricating any Q-Card of the present disclosure can comprise fabricating the first plate or the second plate, using injection molding, laser cutting the first plate, nanoimprinting, extrusion printing, or a combination of thereof.

In certain embodiments of the present disclosure, a method for fabricating any Q-Card of the present disclosure can comprise a step of attaching the hinge on the first and the second plates after the fabrication of the first and second plates.

### ADDITIONAL EXAMPLES

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### (1) Definitions

The terms used in describing the devices/apparatus, systems, and methods herein disclosed are defined in the current application, or in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF card) that regulate the spacing between the plates. The term “X-plate” refers to one of the two plates in a CROF card, wherein the spacers are fixed to this plate. More descriptions of the COF Card, CROF Card, and X-plate are given in the provisional application Ser. Nos. 62/456,065, filed on Feb. 7, 2017, which is incorporated herein in its entirety for all purposes.

### (2) Sample

The devices/apparatus, systems, and methods herein disclosed can be applied to manipulation and detection of various types of samples. The samples are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, US Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for samples such as but not limited to diagnostic samples, clinical samples, environmental samples and foodstuff samples. The types of sample include but are not limited to the samples listed, described and/or summarized in PCT Application (designating U.S.) Nos. PCT/US2016/045437 and PCT/US0216/051775, which were respectively filed on Aug. 10, 2016 and Sep. 14, 2016, and are hereby incorporated by reference by their entireties.

For example, in some embodiments, the devices, apparatus, systems, and methods herein disclosed are used for a sample that includes cells, tissues, bodily fluids and/or a mixture thereof. In some embodiments, the sample comprises a human body fluid. In some embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled breath condensate.

In some embodiments, the devices, apparatus, systems, and methods herein disclosed are used for an environmental sample that is obtained from any suitable source, such as but not limited to: river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. In certain embodiments, the environmental sample is fresh from the source; in certain embodiments, the environmental sample is processed. For example, samples that are not in liquid form are converted to liquid form before the subject devices, apparatus, systems, and methods are applied.

In some embodiments, the devices, apparatus, systems, and methods herein disclosed are used for a foodstuff sample, which is suitable or has the potential to become suitable for animal consumption, e.g., human consumption. In some embodiments, a foodstuff sample includes raw ingredients, cooked or processed food, plant and animal sources of food, preprocessed food as well as partially or fully processed food, etc. In certain embodiments, samples that are not in liquid form are converted to liquid form before the subject devices, apparatus, systems, and methods are applied.

The subject devices, apparatus, systems, and methods can be used to analyze any volume of the sample. Examples of the volumes include, but are not limited to, about 10 mL or less, 5 mL or less, 3 mL or less, 1 microliter (μL, also “μL” herein) or less, 500 μL or less, 300 μL or less, 250 μL or less, 200 μL or less, 170 μL or less, 150 μL or less, 125 μL or less, 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the volume of the sample includes, but is not limited to, about 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values. In some embodiments, the volume of the sample includes, but is not limited to, about 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the amount of the sample is about a drop of liquid. In certain embodiments, the amount of sample is the amount collected from a pricked finger or fingerstick. In certain embodiments, the amount of sample is the amount collected from a microneedle, micropipette or a venous draw.

In certain embodiments, the sample holder is configured to hold a fluidic sample. In certain embodiments, the sample holder is configured to compress at least part of the fluidic sample into a thin layer. In certain embodiments, the sample holder comprises structures that are configured to heat and/or cool the sample. In certain embodiments, the heating source provides electromagnetic waves that can be absorbed by certain structures in the sample holder to change the temperature of the sample. In certain embodiments, the signal sensor is configured to detect and/or measure a signal from the sample. In certain embodiments, the signal sensor is configured to detect and/or measure an analyte in the sample. In certain embodiments, the heat sink is configured to absorb heat from the sample holder and/or the heating source. In certain embodiments, the heat sink comprises a chamber that at least partly enclose the sample holder.

### (3) Q-Card, Spacers and Uniform Sample Thickness

The devices/apparatus, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity. The structure, material, function, variation and dimension of the spacers, as well as the uniformity of the spacers and the sample layer, are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The term “open configuration” of the two plates in a QMAX process means a configuration in which the two plates are either partially or completely separated apart and the spacing between the plates is not regulated by the spacers

The term “closed configuration” of the two plates in a QMAX process means a configuration in which the plates are facing each other, the spacers and a relevant volume of the sample are between the plates, the relevant spacing between the plates, and thus the thickness of the relevant volume of the sample, is regulated by the plates and the spacers, wherein the relevant volume is at least a portion of an entire volume of the sample.

The term “a sample thickness is regulated by the plate and the spacers” in a QMAX process means that for a give condition of the plates, the sample, the spacer, and the plate compressing method, the thickness of at least a port of the sample at the closed configuration of the plates can be predetermined from the properties of the spacers and the plate. The term “inner surface” or “sample surface” of a plate in a QMAX card refers to the surface of the plate that touches the sample, while the other surface (that does not touch the sample) of the plate is termed “outer surface”.

The term “height” or “thickness” of an object in a QMAX process refers to, unless specifically stated, the dimension of the object that is in the direction normal to a surface of the plate. For example, spacer height is the dimension of the spacer in the direction normal to a surface of the plate, and the spacer height and the spacer thickness means the same thing. The term “area” of an object in a QMAX process refers to, unless specifically stated, the area of the object that is parallel to a surface of the plate. For example, spacer area is the area of the spacer that is parallel to a surface of the plate.

The term of QMAX card refers the device that perform a QMAX (e.g. CROF) process on a sample, and have or not have a hinge that connect the two plates.

The term “QMAX card with a hinge and “QMAX card” are interchangeable. The term “angle self-maintain”, “angle self-maintaining”, or “rotation angle self-maintaining” refers to the property of the hinge, which substantially maintains an angle between the two plates, after an external force that moves the plates from an initial angle into the angle is removed from the plates.

In using QMAX card, the two plates need to be open first for sample deposition. However, in some embodiments, the QMAX card from a package has the two plates are in contact each other (e.g. a close position), and to separate them is challenges, since one or both plates are very thing. To facilitate an opening of the QMAX card, opening notch or notches are created at the edges or corners of the first plate or both places, and, at the close position of the plates, a part of the second plate placed over the opening notch, hence in the notch of the first plate, the second plate can be lifted open without a blocking of the first plate.

In the QMAX assay platform, a QMAX card uses two plates to manipulate the shape of a sample into a thin layer (e.g. by compressing). In certain embodiments, the plate manipulation needs to change the relative position (termed: plate configuration) of the two plates several times by human hands or other external forces. There is a need to design the QMAX card to make the hand operation easy and fast.

In QMAX assays, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers. In some embodiments, the average spacing between the two plates is more than 300 um.

In a QMAX assay operation, an operator needs to first make the two plates to be in an open configuration ready for sample deposition, then deposit a sample on one or both of the plates, and finally close the plates into a close position. In certain embodiments, the two plates of a QMAX card are initially on top of each other and need to be separated to get into an open configuration for sample deposition. When one of the plate is a thin plastic film (175 um thick PMA), such separation can be difficult to perform by hand. The present invention intends to provide the devices and methods that make the operation of certain assays, such as the QMAX card assay, easy and fast.

In some embodiments, the QMAX device comprises a hinge that connect two or more plates together, so that the plates can open and close in a similar fashion as a book. In some embodiments, the material of the hinge is such that the hinge can self-maintain the angle between the plates after adjustment. In some embodiments, the hinge is configured to maintain the QMAX card in the closed configuration, such that the entire QMAX card can be slide in and slide out a card slot without causing accidental separation of the two plates. In some embodiments, the QMAX device comprises one or more hinges that can control the rotation of more than two plates.

In some embodiments, the hinge is made from a metallic material that is selected from a group consisting of gold, silver, copper, aluminum, iron, tin, platinum, nickel, cobalt, alloys, or any combination of thereof. In some embodiments, the hinge comprises a single layer, which is made from a polymer material, such as but not limited to plastics. The polymer material is selected from the group consisting of acrylate polymers, vinyl polymers, olefin polymers, cellulosic polymers, noncellulosic polymers, polyester polymers, Nylon, cyclic olefin copolymer (COC), poly(methyl methacrylate) (PMMB), polycarbonate (PC), cyclic olefin polymer (COP), liquid crystalline polymer (LCP), polyamide (PB), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFB), polydimethylsiloxane (PDMS), rubbers, or any combinations of thereof. In some embodiments, the polymer material is selected from polystyrene, PMMB, PC, COC, COP, other plastic, or any combination of thereof.

In essence, the term “spacers” or “stoppers” refers to, unless stated otherwise, the mechanical objects that set, when being placed between two plates, a limit on the minimum spacing between the two plates that can be reached when compressing the two plates together. Namely, in the compressing, the spacers will stop the relative movement of the two plates to prevent the plate spacing becoming less than a preset (i.e. predetermined) value.

The term “a spacer has a predetermined height” and “spacers have a predetermined inter-spacer distance” means, respectively, that the value of the spacer height and the inter spacer distance is known prior to a QMAX process. It is not predetermined, if the value of the spacer height and the inter-spacer distance is not known prior to a QMAX process. For example, in the case that beads are sprayed on a plate as spacers, where beads are landed at random locations of the plate, the inter-spacer distance is not predetermined. Another example of not predetermined inter spacer distance is that the spacers moves during a QMAX processes.

The term “a spacer is fixed on its respective plate” in a QMAX process means that the spacer is attached to a location of a plate and the attachment to that location is maintained during a QMAX (i.e. the location of the spacer on respective plate does not change) process. An example of “a spacer is fixed with its respective plate” is that a spacer is monolithically made of one piece of material of the plate, and the location of the spacer relative to the plate surface does not change during the QMAX process. An example of “a spacer is not fixed with its respective plate” is that a spacer is glued to a plate by an adhesive, but during a use of the plate, during the QMAX process, the adhesive cannot hold the spacer at its original location on the plate surface and the spacer moves away from its original location on the plate surface.

In some embodiments, human hands can be used to press the plates into a closed configuration; In some embodiments, human hands can be used to press the sample into a thin layer. The manners in which hand pressing is employed are described and/or summarized in PCT Application (designating U.S.) Nos. PCT/US2016/045437 filed on Aug. 10, 2016 and PCT/US0216/051775 filed on Sep. 14, 2016, and in U.S. Provisional Application Nos. 62/431,639 filed on Dec. 9, 2016, 62/456,287 filed on Feb. 8, 2017, 62/456,065 filed on Feb. 7, 2017, 62/456,504 filed on Feb. 8, 2017, and 62/460,062 filed on Feb. 16, 2017, which are all hereby incorporated by reference by their entireties.

In some embodiments, human hand can be used to manipulate or handle the plates of the QMAX device. In certain embodiments, the human hand can be used to apply an imprecise force to compress the plates from an open configuration to a closed configuration. In certain embodiments, the human hand can be used to apply an imprecise force to achieve high level of uniformity in the thickness of the sample (e.g. less than 5%, 10%, 15%, or 20% variability).

### (4) Hinges, Opening Notches, Recessed Edge and Sliders

The devices/apparatus, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/431,639, which was filed on Dec. 9, 2016, U.S. Provisional Application No. 62/456,065, which was filed on Feb. 7, 2017, U.S. Provisional Application Nos. 62/456,287 and 62/456,504, which was filed on Feb. 8, 2017, and U.S. Provisional Application No. 62/539,660, which was filed on Aug. 1, 2017, all of which applications are incorporated herein in their entireties for all purposes.

In some embodiments, the QMAX device comprises opening mechanisms such as but not limited to notches on plate edges or strips attached to the plates, making is easier for a user to manipulate the positioning of the plates, such as but not limited to separating the plates of by hand.

In some embodiments, the QMAX device comprises trenches on one or both of the plates. In certain embodiments, the trenches limit the flow of the sample on the plate.

### (5) Q-Card and Adaptor

The devices/apparatus, systems, and methods herein disclosed can include or use Q-cards for sample detection, analysis, and quantification. In some embodiments, the Q-card is used together with an adaptor that is configured to accommodate the Q-card and connect to a mobile device so that the sample in the Q-card can be imaged, analyzed, and/or measured by the mobile device. The structure, material, function, variation, dimension and connection of the Q-card, the adaptor, and the mobile are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application No. 62/460,075 and 62/459,920, which were filed on Feb. 16, 2017, all of which applications are incorporated herein in their entireties for all purposes.

In some embodiments, the adaptor comprises a receptacle slot, which is configured to accommodate the QMAX device when the device is in a closed configuration. In certain embodiments, the QMAX device has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the QMAX device and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample.

### (6) Smartphone Detection System

The devices/apparatus, systems, and methods herein disclosed can include or use Q-cards for sample detection, analysis, and quantification. In some embodiments, the Q-card is used together with an adaptor that can connect the Q-card with a smartphone detection system. In some embodiments, the smartphone comprises a camera and/or an illumination source The smartphone detection system, as well the associated hardware and software are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and US Provisional Application No. 62/460,075 and 62/459,920, which were filed on Feb. 16, 2017, all of which applications are incorporated herein in their entireties for all purposes.

In some embodiments, the smartphone comprises a camera, which can be used to capture images or the sample when the sample is positioned in the field of view of the camera (e.g. by an adaptor). In certain embodiments, the camera includes one set of lenses (e.g. as in iPhone™ 6). In certain embodiments, the camera includes at least two sets of lenses (e.g. as in iPhone™ 7). In some embodiments, the smartphone comprises a camera, but the camera is not used for image capturing.

In some embodiments, the smartphone comprises a light source such as but not limited to LED (light emitting diode). In certain embodiments, the light source is used to provide illumination to the sample when the sample is positioned in the field of view of the camera (e.g. by an adaptor). In some embodiments, the light from the light source is enhanced, magnified, altered, and/or optimized by optical components of the adaptor.

In some embodiments, the smartphone comprises a processor that is configured to process the information from the sample. The smartphone includes software instructions that, when executed by the processor, can enhance, magnify, and/or optimize the signals (e.g. images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

In some embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

In some embodiments, the smartphone is an iPhone™, an Android™ phone, or a Wndows™ phone.

### (7) Detection Methods

The devices/apparatus, systems, and methods herein disclosed can include or be used in various types of detection methods. The detection methods are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, US Provisional Application Nos. 62/456,287, 62/456,528, 62/456,631, 62/456,522, 62/456,598, 62/456,603, and 62/456,628, which were filed on Feb. 8, 2017, U.S. Provisional Application No. 62/459,276, 62/456,904, 62/457,075, and 62/457,009, which were filed on Feb. 9, 2017, and U.S. Provisional Application No. 62/459,303, 62/459,337, and 62/459598, which were filed on Feb. 15, 2017, and U.S. Provisional Application No. 62/460,083, 62/460,076, which were filed on Feb. 16, 2017, all of which applications are incorporated herein in their entireties for all purposes.

### (8) Labels, Capture Agent and Detection Agent

The devices/apparatus, systems, and methods herein disclosed can employ various types of labels, capture agents, and detection agents that are used for analytes detection. The labels are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456065, which was filed on Feb. 7, 2017, U.S. Provisional Application No. 62/456287, which was filed on Feb. 8, 2017, and U.S. Provisional Application No. 62/456,504, which was filed on Feb. 8, 2017, all of which applications are incorporated herein in their entireties for all purposes.

### (9) Analytes

The devices/apparatus, systems, and methods herein disclosed can be applied to manipulation and detection of various types of analytes (including biomarkers). The analytes are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for the detection, purification and/or quantification of various analytes. In some embodiments, the analytes are biomarkers that associated with various diseases. In some embodiments, the analytes and/or biomarkers are indicative of the presence, severity, and/or stage of the diseases. The analytes, biomarkers, and/or diseases that can be detected and/or measured with the devices, apparatus, systems, and/or method of the present invention include the analytes, biomarkers, and/or diseases listed, described and/or summarized in PCT Application (designating U.S.) Nos. PCT/US2016/045437 filed on Aug. 10, 2016, and PCT Application No. PCT/US2016/054025 filed on Sep. 27, 2016, and U.S. Provisional Application Nos. 62/234,538 filed on Sep. 29, 2015, 62/233,885 filed on Sep. 28, 2015, 62/293,188 filed on Feb. 9, 2016, and 62/305,123 filed on Mar. 8, 2016, which are all hereby incorporated by reference by their entireties. For example, the devices, apparatus, systems, and methods herein disclosed can be used in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the analyte can be a biomarker, an environmental marker, or a foodstuff marker. The sample in some instances is a liquid sample, and can be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the sample can be a diagnostic sample obtained from a subject, the analyte can be a biomarker, and the measured the amount of the analyte in the sample can be diagnostic of a disease or a condition.

In any embodiment, the devices, apparatus, systems, and methods in the present invention can further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the biomarker can be selected from Tables B1, 2, 3 or 7 as disclosed in U.S. Provisional Application Nos. 62/234,538, 62/293,188, and/or 62/305,123, and/or PCT Application No. PCT/US2016/054,025, which are all incorporated in their entireties for all purposes. In some instances, the biomarker is a protein selected from Tables B1, 2, or 3. In some instances, the biomarker is a nucleic acid selected from Tables B2, 3 or 7. In some instances, the biomarker is an infectious agent-derived biomarker selected from Table B2. In some instances, the biomarker is a microRNA (miRNA) selected from Table B7.

In any embodiment, the applying step b) can include isolating miRNA from the sample to generate an isolated miRNA sample, and applying the isolated miRNA sample to the disk-coupled dots-on-pillar antenna (QMAX device) array.

In any embodiment, the QMAX device can contain a plurality of capture agents that each bind to a biomarker selected from Tables B1, B2, B3 and/or B7, wherein the reading step d) includes obtaining a measure of the amount of the plurality of biomarkers in the sample, and wherein the amount of the plurality of biomarkers in the sample is diagnostic of a disease or condition.

In any embodiment, the capture agent can be an antibody epitope and the biomarker can be an antibody that binds to the antibody epitope. In some embodiments, the antibody epitope includes a biomolecule, or a fragment thereof, selected from Tables B4, B5 or B6. In some embodiments, the antibody epitope includes an allergen, or a fragment thereof, selected from Table B5. In some embodiments, the antibody epitope includes an infectious agent-derived biomolecule, or a fragment thereof, selected from Table B6.

### (10) Applications

The devices/apparatus, systems, and methods herein disclosed can be used for various applications (fields and samples). The applications are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

### (11) Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/045437 filed on Aug. 10, 2016, and U.S Provisional Application Nos. 62,431,639 filed on Dec. 9, 2016 and 62/456,287 filed on Feb. 8, 2017, which are all hereby incorporated by reference by their entireties.

### (12) Cloud

The devices/apparatus, systems, and methods herein disclosed can employ cloud technology for data transfer, storage, and/or analysis. The related cloud technologies are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the cloud storage and computing technologies can involve a cloud database. Merely by way of example, the cloud platform can include a private cloud, a public cloud, a hybrid cloud, a community cloud, a distributed cloud, an inter-cloud, a multi-cloud, or the like, or any combination thereof. In some embodiments, the mobile device (e.g. smartphone) can be connected to the cloud through any type of network, including a local area network (LAN) or a wide area network (WAN).

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the cloud.

What is claimed:

1. A device for analyzing a thin layer sample, comprising:
a first plate and a diffusion marker,
wherein the first plate has a sample contact area on its inner surface for contacting a thin layer sample of a thickness of 1 mm or less,
wherein the diffusion marker is positioned in the sample contact area of the first plate and is configured to, upon contacting the sample, diffuse in the sample with a pre-determined diffusion rate,
wherein the diffusion marker is distinguishable from the sample when diffusing in the sample, and
wherein the diffusion of the diffusion marker indicates a time duration that the sample is in contact with the first plate inner surface.

2. A device for analyzing a liquid sample, comprising:
a first plate, a second plate, spacers, and a diffusion marker, wherein:
i. the plates are movable relative to each other into different configurations;
ii. one or both plates are flexible;
iii. both plates have, on its respective inner surface, a sample contact area for contacting a sample;
iv. the spacers are fixed to the respective inner surface of one or both of the plates and have a predetermined substantially uniform height;
v. the diffusion marker is positioned in the sample contact area of one or both of the plates and is configured to, upon contacting the sample, diffuse in the sample with a pre-determined diffusion rate; and
vi. the diffusion marker is distinguishable from the sample when diffusing in the sample;
wherein one of the configurations is an open configuration, in which: the two plates are partially or entirely separated apart, the spacing between the plates is not regulated by the spacers, and the sample is deposited on one or both of the plates,
wherein another of the configurations is a closed configuration, which is configured after the sample deposition in the open configuration, and in the closed configuration: at least part of the deposited sample is compressed by the two plates into a layer of uniform thickness that is confined by the two plates, and the uniform thickness of the layer is regulated by the plates and the spacers, wherein the sample thickness is 1 mm or less, and
wherein the diffusion of the diffusion marker indicates a time duration that the sample is in contact with the respective inner surface of the plate on which the diffusion marker is positioned.

3. The device of any prior claim, wherein the diffusion marker is one single entity and diffuses in one direction in the sample, and wherein a diffusion distance of the diffusion marker indicates the time duration that the sample is in contact with the respective plate on which the diffusion marker is positioned.

4. The device of any prior claim, wherein the diffusion marker comprises a plurality of entities that all diffuse in one direction in the sample, and wherein a diffusion distance of the diffusion marker indicates the time duration that the sample is in contact with the respective plate on which the diffusion marker is positioned.

5. The device of any prior claim, wherein the diffusion marker comprises a plurality of entities, and the diffusion marker as a whole diffuses in more than one direction in the sample, and wherein at least one dimension of the diffusion marker indicates the time duration that the sample is in contact with the respective plate on which the diffusion marker is positioned.

6. The device of any prior claim, wherein the diffusion marker diffuses isotropically in the more than one diffusion direction in the liquid solution.

7. The device of any prior claim, wherein the diffusion marker diffuses anisotropically in the more than one diffusion direction in the liquid solution

8. The device of any prior claim, wherein the diffusion marker is confined by a physical barrier thereby preventing the diffusion marker from diffusing in one or more directions in the liquid solution.

9. The device of any prior claim, wherein the diffusion marker is confined to diffuse in a groove on the inner surface of the plate on which the diffusion marker is positioned.

10. The device of any prior claim, wherein the diffusion marker, when diffusing in the sample, is distinguishable from the sample by at least one parameter of the diffusion marker that is selected from the group consisting of light absorption, reflection, transmission, diffraction, scattering, and diffusion; luminescence, heat, viscosity, and magnetism.

11. The device of any prior claim, wherein the predetermined diffusion rate is constant.

12. The device of any prior claim, wherein the predetermined diffusion rate is a predetermined function of time.

13. A method of analyzing a liquid sample, comprising:
(a) obtaining a device of any prior claim;
(b) depositing the liquid sample on one or both of the plates of the device at the open configuration;
(c) bringing the two plates together and compressing the plates into the closed configuration;
(d) at the closed configuration, analyzing the liquid sample at a time point; and
(e) determining at the time point the time duration that the sample is deposited on one or both of the plates by monitoring the diffusion of the diffusion marker in the deposited sample.

14. A device for analyzing a liquid sample, comprising:
a first plate and an interaction marker,
wherein the first plate has a sample contact area on its inner surface for contacting the sample,
wherein the interaction marker is positioned in the sample contact area of the first plate and is configured to, upon contacting the sample, interact with the sample to bring about an interaction signal, and
wherein the interaction signal is configured to indicate the time duration that the sample is in contact with the first plate inner surface.

15. A device for analyzing a liquid sample, comprising:
a first plate, a second plate, spacers, and an interaction marker, wherein:
i. the plates are movable relative to each other into different configurations;
ii. one or both plates are flexible;
iii. both plates have, on its respective inner surface, a sample contact area for contacting a sample;
iv. the spacers are fixed to the respective inner surface of one or both of the plates and have a predetermined substantially uniform height; and
v. the interaction marker is positioned in the sample contact area of one or both of the plates and is configured to, upon contacting the sample, interact with the sample to bring about an interaction signal;
wherein one of the configurations is an open configuration, in which: the two plates are partially or entirely separated apart, the spacing between the plates is not regulated by the spacers, and the sample is deposited on one or both of the plates;
wherein another of the configurations is a closed configuration, which is configured after the sample deposition in the open configuration, and in the closed configuration: at least part of the deposited sample is compressed by the two plates into a layer of uniform thickness that is confined by the two plates, and the uniform thickness of the layer is regulated by the plates and the spacers; and
wherein the interaction signal is configured to indicate the time duration that the sample is in contact with the respective inner surface of the plate on which the interaction marker is positioned.

16. The device of any prior claim, wherein the interaction marker comprises of a plurality of entities of one species.

17. The device of any prior claim, wherein the interaction marker comprises of a plurality of entities of different species, and the entities of different species are located on the inner surfaces of different plates.

18. The device of any prior claim, wherein the interaction signal is provided by the interaction marker.

19. The device of any prior claim, wherein the interaction signal is provided by the sample.

20. The device of any prior claim, wherein the interaction signal is provided by an external entity.

21. The device of any prior claim, wherein the interaction signal is in a signal form selected from the group consisting of: chromatic signal, luminescence signal, heat, magnetic signal, electric signal, sound, any other electromagnetic signals, and any combination thereof.

22. A method of analyzing a liquid sample, comprising:
(a) obtaining a device of any prior claim;
(b) depositing the liquid sample on one or both of the plates of the device at the open configuration;
(c) bringing the two plates together and compressing the plates into the closed configuration;
(d) at the closed configuration, analyzing the liquid sample at a time point; and
(e) determining at the time point the time duration that the sample is deposited on one or both of the plates by monitoring the interaction signal.

23. The device or method of any prior claim, wherein the determining is performed using at least one optical imaging method.

24. The device or method of any prior claim, wherein the layer of uniform thickness limits the diffusion of the interaction marker to 1 dimension or 2 dimensions.

25. The device or method of any prior claim, wherein the interaction marker is a dye, and wherein the dye changes color upon contacting the sample.

26. The device or method of any prior claim, wherein the diffusion marker is an optical label.

27. The device or method of any prior claim, wherein the method is performed under imperfect conditions.

28. The device or method of any prior claim, wherein artificial intelligence and/or machine learning is used to accurately determine the time duration.

29. The device or method of any prior claim, wherein the images taken during an assay operation and/or the samples measured by an assay are analyzed by artificial intelligence and machine learning.

30. The device or method of any prior claim, wherein the samples are selected from the group consisting of medical samples, biology samples, environmental samples and chemistry samples.

31. The device or method of any prior claim, wherein the sample is held by a QMAX device.

32. The device or method of any prior claim, further comprising providing a machine learning framework to enhance the functionality, application scope and/or the accuracy in assaying using QMAX device.

33. A method for assaying a sample that utilizes a QMAX device together with imaging and a machine learning and/or artificial intelligence comprising:
(a) using a QMAX device that has an auxiliary structure in the form of pillars to precisely control the distribution and volume of the sample in assaying, wherein the sample for assaying is loaded into the QMAX device and is kept between the two parallel plates on the QMAX device with an upper plate being transparent for imaging by an imager;
(b) the gap between the two parallel plates in the QMAX device is spaced narrowly—with the distance of the gap being proportional to the size of the analytes to be assayed by which the analytes in the sample form a single layer between the plates that are imaged by an imager on the QMAX device;
(c) the sample volume corresponding to the Aol (area-of-interest) on the upper plate of the QMAX device is precisely characterized by Aol and the gap because of the uniformity of the gap between the plates in the QMAX device;
(d) the image of the sample for assaying sandwiched between the Aol x gap in the QMAX device is a pseudo-2D image, because it has the appearance of a 2D image, but it is an image of a 3D sample with its depth being known priori a priori or characterized through other means;
(e) the captured pseudo-2D sample image taken over the Aol of the QMAX device characterizes the location of the analytes, color, shape, counts, and the concentration of the analytes in the sample for assaying; and
(f) based on abovementioned properties, the captured pseudo-2D image of QMAX device for assaying is amendable to a machine learning framework that applies to analyte detection, localization, identification, segmentation, counting, for assaying in various application.

34. The device or method of any prior claim, further comprising implementing a machine learning framework for QMAX based devices into a device that is capable of running an algorithm such as deep learning to discriminatively locate, identify, segment and count analytes based on the pseudo-2D image captured by the QMAX imager.

35. The device or method of any prior claim, wherein the machine learning improves the images captured by the imager on the QMAX device and reduces the effects of noise and artifacts—including and not limited to air bobbles, dusts, shadows, and pillars.

36. The device or method of any prior claim, wherein the training of machine learning uses the spacers of the QMAX card to reduce the data size of training set.
